(19) United States
(12) Patent Application Publication
(10) Pub. No.: US 2017/0342495 A1
(43) Pub. Date: Nov. 30, 2017

(54) MARKERS OF PRETERM BIRTH

(71) Applicant: Sinai Health System, Toronto (CA)

(72) Inventors: Jan Heng, Allston, MA (US); Stephen Lye, Toronto (CA); Craig Pennell, Floreat (AU)

(21) Appl. No.: 15/591,185

(22) Filed: May 10, 2017

Related U.S. Application Data
(60) Provisional application No. 62/334,744, filed on May 11, 2016.

(51) Int. Cl. C12Q 1/68; G01N 33/68

(52) U.S. Cl. CPC C12Q 1/6883; G01N 33/689; C12Q 2600/158; G01N 2800/368

(57) ABSTRACT

The invention relates to novel markers of preterm birth, methods for assessing the status of preterm birth using the markers, and methods for the diagnosis and therapy of preterm birth.

### FIELD OF THE INVENTION

The invention relates to novel markers of preterm birth, methods for assessing spontaneous preterm birth using the markers, and methods for the detection, diagnosis, prediction, monitoring, preventing, and therapy of preterm birth.

### BACKGROUND OF THE INVENTION

Preterm birth (PTB; birth at <37 weeks of gestation) occurs in about 8-11% of pregnancies worldwide and remains the main cause of perinatal mortality and morbidity in the developed world [Krupa et al, 2006]. Medical advances have increased the survival rates of premature babies; however, premature infants remain vulnerable to disabilities such as respiratory disorders, cognitive impairment, blindness, deafness etc. [Ward and Beachy, 2003]. In later life, they may face complications such as motor and sensory impairment, learning difficulties and behavioral issues. Prematurity leads to an immediate and long term emotional and financial burden to families, communities and the health care system [Petrou, 2005; Institute of Medicine, 2007; Mangham et al, 2009; Canadian Institute for Health Information, 2009; and Lim et al, 2009].

The cornerstone of PTB prevention is identifying asymptomatic women at high risk of early delivery. Current screening tools for spontaneous PTB (SPTB) in asymptomatic women include: clinical risk factor assessment [Dekker et al, 2012; Goldenberg et al, 1998], measuring cervical length with transvaginal ultrasound [Care et al, 2014; Barros-Silva et al, 2013; Resnik, 2005; Iams et al, 1996; Baxter et al, 2014; Hassan et al, 2000]; screening for bacterial vaginosis [Klebanoff et al, 2005; Goffinet et al, 2003]; and measurement of biochemical markers such as phosphorylated insulin-like growth factor binding protein-1 [Kekki et al, 2001; Rutanen et al, 1993; Paternoster et al, 2007] and fetal fibronectin (fFN) [Honest et al, 2002; Chien et al, 1997; Duhig et al, 2009; Leeson et al, 1996; Goldenberg et al, 2000; Sanchez-Ramos et al, 2009; Revah et al, 1998]. These tools are limited by their low sensitivities for SPTB in asymptomatic women that are usually less than 50% with some as low as 8% [Goffinet, 2005; Dekker et al, 2012; Menon et al, 2011].

There is now a wealth of data demonstrating that medical interventions such as progesterone [Fonseca et al, 2007; Meis et al, 2003; Defranco et al, 2007] and cervical cerclage [Alfirevic et al, 2013; Berghella and Keeler, 2010] in asymptomatic high risk women are only beneficial in subgroups of women who have a past history of PTB [Meis et al, 2003] or cervical shortening [Fonseca et al, 2007; Defranco et al, 2007]. The lack of a reliable screening test to help clinicians identify asymptomatic women at risk of PTB has impeded the development and implementation of preventive measures as well as efforts to improve the clinical management of PTB. The serious consequences and complex aetiologies of PTB highlight the need for a multidisciplinary approach to identify the factors that may predict PTB and lead to improved management of women at-risk of PTB [Pennell et al, 2007].

### SUMMARY OF THE INVENTION

Applicants have identified distinct patterns of gene expression in asymptomatic women who eventually had preterm deliveries. In particular, gene expression profiles of asymptomatic women at 17-23 weeks or 27-33 weeks of gestation were different between women who did and did not have a preterm delivery. Temporal gene expression profiles between 17-23 weeks and 27-33 weeks of gestation within the same woman were also predictive of preterm delivery. This early screening test will help clinicians identify asymptomatic women at risk of preterm birth, identify women who will benefit from progesterone, cervical cerclage, or pessary therapies and improve the clinical management of preterm birth.

Broadly stated, the invention provides sets of markers that can identify asymptomatic women who will have preterm deliveries. Methods are provided for use of these markers to distinguish between patient groups, and to determine general courses of treatment. In aspects of the invention, selected markers can identify women at risk of preterm delivery at 17-23 weeks and/or 23-33 weeks of gestation.

The invention provides gene marker sets, biomarker panels or signatures comprising genes or polynucleotides associated with preterm birth in asymptomatic women. A gene marker set, biomarker panel or signature may comprise a plurality of genes comprising or consisting of at least 4, 5, 10, 15, or all of the Preterm Marker Polynucleotides (“PTM Polynucleotides”) disclosed herein. In an aspect, a gene marker set, biomarker panel or signature may comprise a plurality of genes comprising or consisting of at least 4, 5, 10, 15, or all of the genes corresponding to the markers listed in Table 2 [SEQ ID Nos: 1 to 38], 3 or 4. In an aspect the genes correspond to the markers listed in Table 2, 3 and/or 4 for spontaneous preterm birth in asymptomatic women. In an aspect, the gene marker set or signature comprises gene clusters which may be represented by dendrograms or comprise genes in pathways of up and/or down regulated genes listed in Table 2, 3 or 4.

In certain aspects, the plurality of genes comprises, is chosen from or consists of ZNF605, LRRC41, PCDHGA12, ABT1, THBS3, VNN1, LOC100128908, CST13P, EEF1D, RPH3A, TRBV6-6, PLEC, MIR601, ZNF16, MIR3691, LOC101927441, ACAP2, ZNF324, SH3PXD2B and/or TBX21. In certain aspects, the plurality of genes comprises, is chosen from or consists of ZNF605, LRRC41, PCDHGA12, ABT1, THBS3 and/or VNN1. In certain aspects, the plurality of genes comprises, is chosen from or consists of LOC100128908, CST13P, EEF1D, RPH3A, TRBV6-6, PLEC, MIR601 and ZNF16. In certain aspects, the plurality of genes comprises, is chosen from or consists of LOC100128908, MIR3691, LOC101927441, CST13P, ACAP2, ZNF324, SH3PXD2B and/or TBX21.

In embodiments of the invention, the plurality of genes are selected from the group consisting of the genes set forth in Table 2 or 4, which genes are up-regulated biomarkers of spontaneous preterm birth. In embodiments of the invention, the plurality of genes are selected from the group consisting of the genes set forth in Table 2 or 4, which genes are down-regulated biomarkers of spontaneous preterm birth.

The invention also contemplates protein marker sets that distinguish spontaneous preterm birth, the protein marker sets comprising or consisting essentially of at least 4, 5, 10, 15, or all of the polypeptides encoded or expressed by the PTM Polynucleotides disclosed herein (i.e., “PTM Polypeptides”). In an aspect, the invention provides protein marker sets that distinguish spontaneous preterm birth, the protein marker sets comprising or consisting essentially of at least 4, 5, 10, 15 or all of the polypeptides encoded or expressed by the polynucleotides listed in Table 2, 3 or 4. In one aspect the polypeptides are those encoded or expressed by the polynucleotides listed in Table 2, 3 or 4 for spontaneous preterm birth in asymptomatic women. In an aspect the protein marker sets comprise or consist of polypeptide clusters, or polypeptides in pathways comprising the markers. In certain aspects, the polypeptides comprise, are chosen from or consist of the polypeptides encoded or expressed by the gene marker sets or signatures disclosed herein.

PTM Polynucleotides associated with spontaneous preterm birth disclosed herein, including the markers listed in Table 2, 3 or 4, and polypeptides encoded or expressed from the PTM Polynucleotides, have application in the detection of spontaneous preterm birth. Thus, the markers can be used for diagnosis, monitoring (i.e. monitoring progression or therapeutic treatment), preventing, prognosis, treatment, or classification of spontaneous preterm birth or as markers before or after therapy.

The levels of PTM Polypeptides or PTM Polynucleotides in a sample may be determined by methods as described herein and generally known in the art. In accordance with methods of the invention, susceptibility to spontaneous preterm birth can be assessed or characterized, for example by detecting or identifying the presence in the sample of (a) a PTM Polypeptide or fragment thereof; (b) a metabolite which is produced directly or indirectly by a PTM Polypeptide; (c) a transcribed polynucleotide or fragment thereof having at least a portion with which a PTM Polynucleotide is substantially identical; and/or (d) a transcribed polynucleotide or fragment thereof, wherein the polynucleotide hybridizes with a PTM Polynucleotide.

In an aspect, the invention provides a method for characterizing or classifying a sample as preterm birth in asymptomatic women comprising detecting a difference in the expression of a first plurality of genes relative to a control, the first plurality of genes consisting of at least 5, 10, 15, or all of the genes corresponding to the PTM Polynucleotides disclosed herein. In a particular aspect, the control comprises polynucleotides derived from a pool of samples from individual term patients.

In an aspect, a method is provided for characterizing susceptibility to spontaneous preterm birth by detecting PTM Polypeptides or PTM Polynucleotides in a subject comprising:
(a) detecting or identifying in a sample from the subject PTM Polypeptides or PTM Polynucleotides; and
(b) comparing the detected amount with an amount detected for a standard.

In an embodiment, the invention provides a method for detecting spontaneous preterm birth in a subject comprising: (a) subjecting a sample from the subject to a procedure to detect PTM Polynucleotides or PTM in the sample; and (b) detecting spontaneous preterm birth by comparing the amount of PTM Polynucleotides or PTM Polypeptides to the amount of the PTM Polynucleotides or PTM Polypeptides obtained from a control.

In an aspect, the invention provides a method of assessing whether a patient has a pre-disposition for preterm birth comprising comparing:
(a) levels of PTM Polypeptides or PTM Polynucleotides in a sample from the patient; and
(b) normal levels of PTM Polypeptides or PTM Polynucleotides in samples of the same type obtained from control patients who delivered to term, wherein altered levels of the PTM Polypeptides or PTM Polynucleotides relative to the corresponding normal levels of the PTM Polypeptides or PTM Polynucleotides is an indication that the patient has a predisposition to preterm birth.

In an embodiment of a method of the invention for assessing whether a patient has a pre-disposition for spontaneous preterm birth, higher levels of PTM Polypeptides or PTM Polynucleotides in a sample relative to the corresponding normal levels is an indication that the patient has a pre-disposition for spontaneous preterm birth. In a particular embodiment the PTM Polynucleotides comprise, are chosen from or consist of the up-regulated genes listed on Table 2 or 4.

In another particular embodiment of a method of the invention for assessing whether a patient has a pre-disposition for spontaneous preterm birth, lower levels of PTM Polypeptides or PTM Polynucleotides in a sample relative to the corresponding normal levels is an indication that the patient has a pre-disposition for spontaneous preterm birth. In a particular embodiment the PTM Polynucleotides comprise, are chosen from or consist of the down-regulated genes listed on Table 2 or 4.

In an embodiment of the invention, a method for screening or monitoring a subject for spontaneous preterm birth is provided comprising (a) detecting the amount of PTM Polypeptides or PTM Polynucleotides associated with spontaneous preterm birth in a biological sample from the subject; and (b) comparing said amount of PTM Polypeptides or PTM Polynucleotides detected to a predetermined standard, where detection of a level of PTM Polypeptides or PTM Polynucleotides that differs significantly from the standard indicates spontaneous preterm birth. In one aspect the PTM Polynucleotides comprise, are chosen from or consist of the genes in Table 2, 3 or 4 for screening or monitoring a subject for spontaneous preterm birth in asymptomatic women. In one aspect the PTM Polypeptides comprise, are chosen from or consist of the polypeptides encoded by or expressed by the genes in Table 2, 3 or 4, for screening or monitoring a subject for spontaneous preterm birth in asymptomatic women.

In an embodiment the amount of PTM Polypeptide(s) or PTM Polynucleotide(s) detected may be greater than that of a standard and is indicative of spontaneous preterm birth. In another embodiment the amount of PTM Polypeptide(s) or PTM Polynucleotide(s) detected is lower than that of a standard and is indicative of spontaneous preterm birth.

An aspect of the invention provides a method of diagnosing spontaneous preterm birth in a patient comprising determining the status of one or more PTM Polynucleotide or PTM Polypeptide in a sample obtained from the patient, wherein an abnormal status of the PTM Polynucleotide or PTM Polypeptide indicates spontaneous preterm birth.

In an aspect, the invention provides a method for detecting the amount of each biomarker in a panel of biomarkers in a sample from a subject and optionally, in a control sample, said method comprising contacting said panel of biomarkers with a reagent specific to each biomarker in said panel of biomarkers and detecting the binding of the reagent specific to each biomarker of the panel of biomarkers, wherein the panel of biomarkers comprises (a) ZNF605, LRRC41, PCDHGA12, ABT1, THBS3, VNN1, LOC100128908, CST13P, EEF1D, RPH3A, TRBV6-6, PLEC, MIR601, ZNF16, MIR3691, LOC101927441, ACAP2, ZNF324, SH3PXD2B and TBX2; (b) ZNF605, LRRC41, PCDHGA12, ABT1, THBS3 and VNN1, (c). LOC100128908, CST13P, EEF1D, RPH3A, TRBV6-6, PLEC, MIR601 and ZNF16, or (d) LOC100128908, MIR3691, LOC101927441, CST13P, ACAP2, ZNF324, SH3PXD2B and TBX21, or polypeptides encoded by (a), (b), (c), or (d).

A method of diagnosing or monitoring spontaneous preterm birth in a subject is provided comprising obtaining a biological sample from the subject, identifying PTM Polynucleotides in the sample associated with spontaneous preterm birth to identify spontaneous preterm birth of a particular etiology, and providing an individualized therapeutic strategy based on the etiology of spontaneous preterm birth identified.

In one aspect the invention provides a method for determining preterm birth in a patient at risk for the development of preterm birth comprising the steps of determining the concentration of one or more markers comprising, chosen from or consisting of the PTM Polynucleotides, in particular the markers in Table 3 or 4.

In an aspect, a method is provided for diagnosing spontaneous preterm birth in an asymptomatic subject comprising comparing the concentration of markers comprising, chosen from or consisting of ZNF605, LRRC41, PCDHGA12, ABT1, THBS3, VNN1, LOC100128908, CST13P, EEF1D, RPH3A, TRBV6-6, PLEC, MIR601, ZNF16, MIR3691, LOC101927441, ACAP2, ZNF324, SH3PXD2B, and TBX21 or polypeptides encoded by same in a sample (e.g. whole blood, serum or plasma) from the subject to a cut-off concentration and determining spontaneous preterm birth development potential from the comparison, wherein significant differences in concentrations of markers are predictive of (e.g., correlate with) spontaneous preterm birth in the subject.

In an aspect, a method is provided for diagnosing spontaneous preterm birth in an asymptomatic subject, the method comprising measuring the level of each biomarker of a panel of biomarkers in a sample from the subject, wherein each biomarker of the panel of biomarkers is measured using a respective reagent that specifically measures the biomarker and the panel of biomarkers comprises (a) ZNF605, LRRC41, PCDHGA12, ABT1, THBS3, VNN1, LOC100128908, CST13P, EEF1D, RPH3A, TRBV6-6, PLEC, MIR601, ZNF16, MIR3691, LOC101927441, ACAP2, ZNF324, SH3PXD2B and TBX2; (b) ZNF605, LRRC41, PCDHGA12, ABT1, THBS3 and VNN1, (c). LOC100128908, CST13P, EEF1D, RPH3A, TRBV6-6, PLEC, MIR601 and ZNF16, or (d) LOC100128908, MIR3691, LOC101927441, CST13P, ACAP2, ZNF324, SH3PXD2B and TBX21, or polypeptides encoded by (a), (b), (c), or (d).

In an aspect, a method is provided for assessing spontaneous preterm birth development potential in an asymptomatic subject comprising comparing the concentration of markers comprising, chosen from or consisting of ZNF605, LRRC41,PCDHGA12, ABT1, THBS3 and/or VNN1, or polypeptides encoded by ZNF605, LRRC41, PCDHGA12, ABT1, THBS3 and VNN1 in a sample (e.g. whole blood, serum or plasma) from the subject to a cut-off concentration and determining spontaneous preterm birth development potential from the comparison, wherein significant differences in concentrations of markers are predictive of (e.g., correlate with) spontaneous preterm birth development in the subject.

In an aspect, a method is provided for assessing spontaneous preterm birth development potential in an asymptomatic subject comprising comparing the concentration of markers comprising, chosen from or consisting of LOC100128908, CST13P, EEF1D, RPH3A, TRBV6-6, PLEC, MIR601, and/or ZNF16, or polypeptides encoded by LOC100128908, CST13P, EEF1D, RPH3A, TRBV6-6, PLEC, MIR601, and/or ZNF16 in a sample (e.g. whole blood, serum or plasma) from the subject to a cut-off concentration and determining spontaneous preterm birth development potential from the comparison, wherein significant differences in concentrations of markers are predictive of (e.g., correlate with) spontaneous preterm birth development in the subject.

In an aspect, a method is provided for assessing spontaneous preterm birth development potential in an asymptomatic subject comprising comparing the concentration of markers comprising, chosen from or consisting of LOC100128908, MIR3691, LOC101927441, CST13P, ACAP2, ZNF324, SH3PXD2B and/or TBX21, or polypeptides encoded by LOC100128908, MIR3691, LOC101927441, CST13P, ACAP2, ZNF324, SH3PXD2B and/or TBX21 in a sample (e.g. whole blood, serum or plasma) from the subject to a cut-off concentration and determining spontaneous preterm birth development potential from the comparison, wherein significant differences in concentrations of markers are predictive of (e.g., correlate with) spontaneous preterm birth development in the subject.

In an aspect, a method is provided for assessing spontaneous preterm birth development potential in an asymptomatic subject less than 23 weeks, or between 17 and 23 weeks, of gestation comprising comparing the concentration of markers comprising, chosen from or consisting of ZNF605, LRRC41, PCDHGA12, ABT1, THBS3 and VNN1, or polypeptides encoded by ZNF605, LRRC41, PCDHGA12, ABT1, THBS3 and VNN1, in a sample (e.g. whole blood, serum or plasma) from the subject to a cut-off concentration and determining preterm development potential from the comparison, wherein significant differences in concentrations of markers are predictive of (e.g., correlate with) spontaneous preterm birth development in the subject.

In an aspect, a method is provided for assessing spontaneous preterm birth development potential in an asymptomatic subject less than 33 weeks, or between 27 and 33 weeks, of gestation comprising comparing the concentration of markers comprising, chosen from or consisting of LOC 100128908, CST13P, EEF1D, RPH3A, TRBV6-6, PLEC, MIR601, and/or ZNF16, or polypeptides encoded by LOC100128908, CST13P, EEF1D, RPH3A, TRBV6-6, PLEC, MIR601, and/or ZNF16 in a sample (e.g. whole blood, serum or plasma) from the subject to a cut-off concentration and determining preterm birth development potential from the comparison, wherein significant differences in concentrations of markers are predictive of (e.g., correlate with) preterm birth development in the subject.

In an aspect, a method is provided for assessing spontaneous preterm birth development potential in an asymptomatic subject comprising comparing the change in concentration of markers comprising, chosen from or consisting of LOC100128908, MIR3691, LOC101927441, CST13P, ACAP2, ZNF324, SH3PXD2B and/or TBX21, or polypeptides encoded by LOC100128908, MIR3691, LOC101927441, CST13P, ACAP2, ZNF324, SH3PXD2B and/or TBX21 in a sample (e.g. whole blood, serum or plasma) from the subject at 17 to 23 weeks (T1) and 27 to 33 weeks (T2) of gestation and determining spontaneous preterm birth development potential from the comparison, wherein significant differences in concentrations of markers are predictive of (e.g., correlate with) spontaneous preterm birth development in the subject.

In an aspect, a method is provided for assessing spontaneous preterm birth development potential in an asymptomatic subject comprising assaying markers comprising, chosen from or consisting of LOC100128908, MIR3691, LOC101927441, CST13P, ACAP2, ZNF324, SH3PXD2B and/or TBX21, or polypeptides encoded by LOC100128908, MIR3691, LOC101927441, CST13P, ACAP2, ZNF324, SH3PXD2B and/or TBX21 in a sample (e.g. whole blood, serum or plasma) from the subject at 17 to 23 weeks (T1) and at 27 to 33 weeks (T2) of gestation and determining spontaneous preterm birth development potential based on the differences in concentrations of markers between T1 and T2.

In aspects of the methods of the invention, the methods are non-invasive for detecting spontaneous preterm birth, which in turn allow for diagnosis of a variety of conditions or diseases associated with such spontaneous preterm birth.

In particular, the invention provides a non-invasive non-surgical method for detection, diagnosis, monitoring, or prediction of preterm birth in a pregnant female comprising: obtaining a sample of blood, plasma, serum, urine or saliva or a tissue sample from the pregnant female; subjecting the sample to a procedure to detect PTM Polypeptide(s) or PTM Polynucleotide(s) in the blood, plasma, serum, urine, saliva or tissue; detecting, diagnosing, and predicting term or spontaneous preterm birth by comparing the levels of PTM Polypeptide(s) or PTM Polynucleotide(s) to the levels of PTM Polypeptide(s) or PTM Polynucleotide(s) obtained from control or from an earlier sample of the pregnant female.

In an embodiment, preterm birth is detected, diagnosed, or predicted by determination of decreased levels of PTM Polynucleotides or PTM Polypeptides when compared to such levels obtained from term delivery controls. In an embodiment, the PTM Polynucleotides comprise, are chosen from or consist essentially of the down-regulated markers listed in Table 2 or 4 or PTM Polypeptides encoded by same.

In another embodiment, preterm birth is detected, diagnosed, or predicted by determination of increased levels of PTM Polynucleotides or PTM Polypeptides when compared to such levels obtained from term delivery controls. In an embodiment, the PTM Polynucleotides comprise, are chosen from or consist essentially of the up-regulated markers listed in Table 2 or 4 or PTM Polypeptides encoded by same.

The invention provides a method for detecting, in particular monitoring, spontaneous preterm birth in a patient the method comprising:
(a) detecting PTM Polypeptides or PTM Polynucleotides in a sample from the patient at a first time point;
(b) repeating step (a) at a subsequent point in time; and
(c) comparing the levels detected in (a) and (b), and therefrom monitoring the spontaneous preterm birth.

In an embodiment of this method of the invention, the first time point is between 17 to 23 weeks of gestation and the subsequent time point is between 27 to 33 weeks of gestation, and the markers comprise, are chosen from or consist of LOC100128908, MIR3691, LOC101927441, CST13P, ACAP2, ZNF324, SH3PXD2B and/or TBX21.The method may further comprise clinical factors including history of abortion and anaemia.

A method of the invention for assessing or detecting preterm birth in a subject may also comprise treating the subject. A treatment includes but is not limited to therapeutics, procedures and interventions such as progesterone, cervical cerclage or pessary.

The invention also provides a method for assessing the potential efficacy of a test agent for preventing, inhibiting, or reducing spontaneous preterm birth and a method of selecting an agent for inhibiting spontaneous preterm birth.

The invention also contemplates a method of assessing the potential of a test agent to contribute to spontaneous preterm birth comprising:
(a) maintaining separate aliquots of samples from a patient in the presence and absence of the test agent; and
(b) comparing the levels of PTM Polypeptides or PTM Polynucleotides in each of the aliquots.

A significant difference between the levels of PTM Polypeptides or PTM Polynucleotides in an aliquot maintained in the presence of (or exposed to) the test agent relative to the aliquot maintained in the absence of the test agent, indicates that the test agent potentially contributes to spontaneous preterm birth.

A method for determining the effect of an environmental factor on spontaneous preterm birth comprising comparing PTM Polynucleotides or PTM Polypeptides associated with spontaneous preterm birth in the presence and absence of the environmental factor.

The invention further relates to a method of assessing the efficacy of a therapy for preventing, inhibiting, or reducing spontaneous preterm birth in a patient. A method of the invention comprises comparing: (a) levels of PTM Polypeptides or PTM Polynucleotides in a sample from the patient obtained from the patient prior to providing at least a portion of a therapy to the patient; and (b) levels of PTM Polypeptides or PTM Polynucleotides in a second sample obtained from the patient following therapy. A significant difference between the levels of PTM Polypeptides or PTM Polynucleotides in the second sample relative to the first sample is an indication that the therapy may be efficacious for inhibiting spontaneous preterm birth. In an embodiment, the method is used to assess the efficacy of a therapy for inhibiting spontaneous preterm birth where lower levels of PTM Polypeptides or PTM Polynucleotides relative to the first sample, is an indication that the therapy may be efficacious for inhibiting the condition. In an embodiment, the method is used to assess the efficacy of a therapy for inhibiting spontaneous preterm birth where higher levels of PTM Polypeptides or PTM Polynucleotides relative to the first sample, is an indication that the therapy may be efficacious for inhibiting spontaneous preterm birth. A “therapy” may be any therapy for treating spontaneous preterm birth, in particular, including but not limited to therapeutics, procedures and interventions such as progesterone, cervical cerclage and pessary. A method of the invention can be used to evaluate a patient before, during, and after therapy.

Methods for diagnosing, detecting or monitoring spontaneous preterm birth are contemplated comprising detecting PTM Polynucleotides associated with preterm birth. Thus, the present invention relates to a method for diagnosing and monitoring spontaneous preterm birth in a sample from a subject comprising isolating polynucleotides, in particular mRNA, from the sample; and detecting PTM Polynucleotides in the sample. The presence of different levels of PTM Polynucleotides in the sample compared to a standard or control may be indicative of spontaneous preterm birth and/or a positive prognosis. In an embodiment of the invention, PTM Polynucleotide positive samples (e.g. higher levels of PTM Polynucleotides compared to a normal control) are a negative diagnostic indicator. Positive samples can be indicative of spontaneous preterm birth or a poor prognosis. In another embodiment of the invention, PTM Polynucleotide negative samples (e.g. lower levels of the PTM Polynucleotides compared to a normal control) are a negative diagnostic indicator. Negative samples can be indicative of spontaneous preterm birth or poor prognosis.

The invention provides methods for determining the presence or absence of spontaneous preterm birth in a subject comprising detecting in the sample levels of polynucleotides that hybridize to one or more PTM Polynucleotides, comparing the levels with a predetermined standard or cut-off value, and therefrom determining the presence or absence of spontaneous preterm birth in the subject. In an embodiment, the invention provides methods for determining the presence or absence of spontaneous preterm birth in a subject comprising (a) contacting a sample obtained from the subject with oligonucleotides that hybridize to one or more PTM Polynucleotides; and (b) detecting in the sample a level of polynucleotides that hybridize to the PTM Polynucleotides relative to a predetermined cut-off value, and therefrom determining the presence or absence of spontaneous preterm birth in the subject.

Within certain embodiments, the amount of polynucleotides that are mRNA are detected via polymerase chain reaction using, for example, oligonucleotide primers that hybridize to one or more PTM Polynucleotides, or complements of such polynucleotides. Within other embodiments, the amount of mRNA is detected using a hybridization technique, employing oligonucleotide probes that hybridize to one or more PTM Polynucleotides, or complements thereof.

When using mRNA detection, the method may be carried out by combining isolated mRNA with reagents to convert to cDNA according to standard methods; treating the converted cDNA with amplification reaction reagents (such as cDNA polymerase chain reaction (PCR) reaction reagents) in a container along with an appropriate mixture of nucleic acid primers; reacting the contents of the container to produce amplification products; and analyzing the amplification products to detect the presence of one or more PTM Polynucleotides in the sample. For mRNA, the analyzing step may be accomplished using Northern Blot analysis to detect the presence of PTM Polynucleotides. The analysis step may be further accomplished by quantitatively detecting the presence of PTM Polynucleotides in the amplification product, and comparing the quantity of markers detected against a panel of expected values for the known presence or absence of the markers in normal tissue derived using similar primers.

The invention provides a method wherein mRNA is detected by (a) isolating mRNA from a sample and combining the mRNA with reagents to convert it to cDNA; (b) treating the converted cDNA with amplification reaction reagents and nucleic acid primers that hybridize to one or more PTM Polynucleotides to produce amplification products; (d) analyzing the amplification products to detect an amount of mRNA encoding the PTM Polypeptides; and (e) comparing the amount of mRNA to an amount detected against a panel of expected values for normal tissue derived using similar nucleic acid primers.

In particular aspects of the invention, the methods described herein utilize the PTM Polynucleotides placed on a microarray so that the expression status of each of the markers is assessed simultaneously.

In an embodiment, the invention provides a preterm marker microarray comprising a defined set of genes whose expression is significantly altered by spontaneous preterm birth. The invention further relates to the use of the microarray as a prognostic tool to predict spontaneous preterm birth. In an embodiment, the invention provides for oligonucleotide arrays comprising marker sets described herein. In an aspect, the microarrays of the present invention comprise probes to distinguish preterm birth. In particular, the invention provides oligonucleotide arrays comprising probes to a subset or subsets of at least 5, 10, 15 or 20 gene markers (e.g. PTM Polynucleotides) up to a full set of markers which distinguish preterm birth patients or samples. In an embodiment, the microarray comprises or consists of the markers in Table 2, 3 or 4. In embodiments, the microarray comprises or consists of the each of the embodiments of the plurality of genes disclosed herein.

Preterm birth may be assessed by determining the levels of specific proteins expressed from PTM Polynucleotides (i.e. the levels of the PTM Polypeptides). Certain methods of the invention employ binding agents (e.g. antibodies) that specifically recognize PTM Polypeptides.

In an embodiment, the invention provides methods for determining spontaneous preterm birth in a patient, comprising the steps of (a) contacting a biological sample obtained from a patient with one or more binding agent that specifically binds to one or more PTM Polypeptides associated with spontaneous preterm birth; and (b) detecting in the sample amounts of markers that binds to the binding agent, relative to a predetermined standard or cut-off value, and therefrom determining the presence or absence of spontaneous preterm birth in the patient.

In another embodiment, the invention relates to a method for diagnosing and monitoring preterm birth in a subject by quantitating one or more PTM Polypeptides associated with preterm birth in a biological sample from the subject comprising (a) reacting the biological sample with one or more binding agent specific for the PTM Polypeptides (e.g. an antibody) that are directly or indirectly labeled with a detectable substance; and (b) detecting the detectable substance.

In an embodiment of the invention, the agent is an antibody which recognizes a PTM Polypeptide. In another embodiment of the invention the agent is a chemical entity which recognizes a PTM Polypeptide. An agent may carry a label or detectable substance to image a PTM Polypeptide and optionally other markers. Examples of labels useful for imaging are radiolabels, fluorescent labels (e.g. fluorescein and rhodamine), nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed.

In an aspect the invention provides a method for using an antibody to detect expression of one or more PTM Polypeptide in a sample, the method comprising: (a) combining antibodies specific for one or more PTM Polypeptide with a sample under conditions which allow the formation of antibody:marker complexes; and (b) detecting complex formation, wherein complex formation indicates expression of the marker in the sample. Expression may be compared with standards and is diagnostic of spontaneous preterm birth.

PTM Polypeptides levels can be determined by constructing an antibody microarray in which binding sites comprise immobilized, preferably monoclonal, antibodies specific to a substantial fraction of marker-derived proteins of interest.

The invention also relates to kits for carrying out the methods of the invention. In an embodiment, the kit is for diagnosing or monitoring spontaneous preterm birth and it comprises reagents for assessing one or more PTM Polypeptides or PTM Polynucleotides. In another embodiment, the invention provides diagnostic tools, and kits for detecting, diagnosing, and predicting the presence or impending onset of spontaneous preterm birth by monitoring levels of PTM Polypeptides or PTM Polynucleotides.

The invention further provides kits comprising the gene or protein marker sets described herein. In an aspect the kit contains a microarray ready for hybridization to target PTM Polynucleotides, plus software for the data analyses.

The invention also provides a diagnostic composition comprising a PTM Polypeptide or a PTM Polynucleotide. A composition is also provided comprising a probe that specifically hybridizes to PTM Polynucleotides, or a fragment thereof, or an antibody specific for a PTM Polypeptide or a fragment thereof In another aspect, a composition is provided comprising one or more PTM Polynucleotide specific primer pairs capable of amplifying the polynucleotides using polymerase chain reaction methodologies. The probes, primers or antibodies can be labeled with a detectable substance.

The invention contemplates the methods, compositions, and kits described herein comprising assessing one or more additional clinical factor or prognostic factor associated with spontaneous preterm birth. The additional factor may be additional markers of spontaneous preterm birth and/or clinical blood data. In an aspect the additional marker is fetal fibronection or phosphorylated insulin-like growth factor binding protein-1. The additional factor may be clinical factors comprising or chosen from or selected from the group consisting of history of abortion, history of PTB, alcohol consumption, anaemia, antepartum haemorrhage and urinary tract infection. The additional factor may be clinical factors comprising or chosen from or selected from the group consisting of history of abortion, history of PTB, alcohol consumption and urinary tract infection. Accordingly, the methods of this invention may be used in combination with other methods of preterm birth diagnosis or clinical factors including without limitation, clinical blood data, fetal fibronectin, phosphorylated insulin-like growth factor binding protein-1, and at least one of history of abortion, history of PTB, alcohol consumption, anaemia, antepartum haemorrhage and urinary tract infection, in particular history of abortion, history of PTB, alcohol consumption, and anaemia. Methods including additional markers can include reagents to detect the additional markers. In an aspect, the methods of this invention are used in combination with the clinical factors history of PTB, history of abortion, and anaemia. In an aspect, the methods of this invention are used in combination with the clinical factors history of PTB and history of abortion. In an aspect, the methods of this invention are used in combination with the clinical factors history of PTB, history of abortion, alcohol consumption, urinary tract infections and anaemia. In an aspect, the methods of this invention are used in combination with the clinical factors history of PTB, history of abortion and urinary tract infections, and optionally anaemia. In an aspect, the methods of this invention are used in combination with the clinical factors history of PTB, history of abortion and alcohol consumption. In an aspect, the methods of this invention are used in combination with the clinical factors history of abortion and anaemia.

In an aspect, a method is provided for assessing spontaneous preterm birth development potential in an asymptomatic subject less than 23 weeks, or between 17 and 23 weeks, of gestation comprising (a) comparing the concentration of markers comprising, chosen from or consisting of ZNF605, LRRC41, PCDHGA12, ABT1, THBS3 and VNN1, or polypeptides encoded by ZNF605, LRRC41, PCDHGA12, ABT1, THBS3 and VNN1, in a sample taken from the subject less than 23 weeks, or between 17 and 23 weeks, of gestation, to a control; (b) assessing clinical factors selected or chosen from history of preterm birth and history of abortion; and optionally alcohol consumption and/or urinary tract infection and (c) determining preterm development potential based on significant differences in concentrations of the markers and the clinical factors to assess spontaneous preterm birth development in the subject.

In an aspect, a method is provided for assessing spontaneous preterm birth development potential in an asymptomatic subject less than 33 weeks, or between 27 and 33 weeks, of gestation comprising (a) comparing the concentration of markers comprising, chosen from or consisting of LOC100128908, CST13P, EEF1D, RPH3A, TRBV6-6, PLEC, MIR601, and/or ZNF16, or polypeptides encoded by LOC100128908, CST13P, EEF1D, RPH3A, TRBV6-6, PLEC, MIR601, and/or ZNF16 in a sample taken from the subject less than 33 weeks, or between 27 and 33 weeks, of gestation, to a control; (b) assessing clinical factors selected or chosen from history of abortion and anaemia; and optionally history of preterm birth, urinary tract infection and/or alcohol consumption; and (c) determining preterm birth development potential based on significant differences in concentrations of the markers and the clinical factors to assess spontaneous preterm birth development in the subject.

In an aspect, a method is provided for assessing spontaneous preterm birth development potential in an asymptomatic subject the method comprising:
(a) assaying the concentration of markers comprising, chosen from or consisting of LOC100128908, MIR3691, LOC101927441, CST13P, ACAP2, ZNF324, SH3PXD2B and/or TBX21 or polypeptides encoded by LOC100128908, MIR3691, LOC101927441, CST13P, ACAP2, ZNF324, SH3PXD2B and/or TBX21 in a sample from the subject between 17 to 23 weeks of gestation;
(b) repeating step (a) in a sample from the subject between 27 to 33 weeks of gestation;
(c) assessing clinical factors selected or chosen from history of abortion and anaemia and optionally alcohol consumption, urinary tract infection and/or history of preterm birth; and
(d) assessing spontaneous preterm birth development potential based on significant differences in concentrations of markers detected in (a) and (b) and the clinical factors to assess spontaneous preterm birth development potential in the subject.

In embodiments of the invention the methods, compositions and kits use one or more of the PTM Polypeptides or PTM Polypeptides. In embodiments of the invention the methods, compositions and kits use one or more of the markers listed in Table 2, 3 or 4. In one aspect the markers correspond to the markers listed in Table 2, 3 or 4 associated with preterm birth. In another embodiment, the methods use a panel of markers comprising or selected from the markers listed in Table 2, 3 or 4, in particular a panel comprising two, three, four, five, six, seven, eight or ten, or more of the markers in Table 2, 3 or 4. In embodiments, the panel comprises, is chosen from or consists of each of the embodiments of the plurality of genes disclosed herein.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIG. 1 is a flowchart outlining the recruitment, patient phenotyping and sample selection process for the study described in the Example herein.

FIG. 2 are area under receiver operator characteristic curves of Models A, B and C after ten five-fold cross-validation (CV) runs. These three multivariate models were constructed to identify gene expression associated with spontaneous preterm birth (SPTB) at 17-23 weeks (A; Model A) and 27-33 weeks (B; Model B); and gene expression fold change between 17-23 and 27-33 weeks of gestation associated with SPTB (C; Model C). Models with clinical factors are represented using solid lines; Models without clinical factors are represented using dotted lines. The rainbow bar on the right of each plot displays cut-off probabilities. The colour of the points along the average CV curve reflects its respective cut-off probability to obtain the desired sensitivity and specificity.

### DETAILED DESCRIPTION OF THE INVENTION

Methods are provided for detecting the presence of spontaneous preterm birth in a sample, the absence of spontaneous preterm birth, and other characteristics of spontaneous preterm birth that are relevant to prevention, diagnosis, monitoring, characterization, and therapy of spontaneous preterm birth in a patient. Methods are also provided for assessing the efficacy of one or more test agents for preventing, inhibiting, or reducing spontaneous preterm birth, assessing the efficacy of a therapy for spontaneous preterm birth, monitoring the progression of pregnancy that results in spontaneous preterm birth, selecting an agent or therapy for spontaneous preterm birth, treating a patient afflicted with spontaneous preterm birth, preventing, inhibiting, or reducing spontaneous preterm birth in a patient, and assessing the potential of a test compound to cause spontaneous preterm birth. In one embodiment, the invention provides a method of using gene expression profiles from whole blood. In one embodiment, the invention provides a method of using gene expression profiles from peripheral blood cells or decidual cells of symptomatic women to predict preterm birth. In an embodiment, the invention provides a method of using gene expression profiles from peripheral blood cells or decidual cells of asymptomatic women to predict preterm deliveries. In an embodiment, the invention provides a method of using gene expression profiles from subpopulations of leukocytes (e.g., macrophages, lymphocytes).

Unless defined otherwise, all technical and scientific terms used herein have the same meaning as commonly understood by one of ordinary skill in the art to which this invention belongs. The following definitions supplement those in the art and are directed to the present application and are not to be imputed to any related or unrelated case. Generally, nomenclatures used in connection with, and techniques of, molecular biology, immunology, microbiology, genetics, protein and nucleic acid chemistry and hybridization described herein are those well-known and commonly used in the art. Methods and techniques employed in the present invention are generally performed according to conventional methods known in the art and as described, for example, in general references such as Sambrook et al, Molecular Cloning: A Laboratory Manual, 2nd ed., Cold Spring Harbor Laboratory Press, Cold Spring Harbor, N.Y. (1989) and Ausubel et al, Current Protocols in Molecular Biology, Greene Publishing Associates (1992) and Harlow and Lane, Antibodies: A Laboratory Manual, Cold Spring Harbor Laboratory Press, Cold Spring Harbor, N.Y., (1990). Although any methods and materials similar or equivalent to those described herein can be used in the practice of the invention, particular materials and methods are described herein.

“Binding agent” refers to a substance such as a polypeptide or antibody that specifically binds to one or more PTM Polypeptide, or in some cases a PTM Polynucleotide. A substance “specifically binds” to one or more PTM Polypeptide if is reacts at a detectable level with one or more PTM Polypeptide, and does not react detectably with peptides containing an unrelated or different sequence. Binding properties may be assessed using an ELISA, which may be readily performed by those skilled in the art (see for example, Newton et al, Develop. Dynamics 197: 1-13, 1993).

A binding agent may be a ribosome, with or without a peptide component, an aptamer, an RNA molecule, or a polypeptide. A binding agent may be a polypeptide that comprises one or more PTM Polypeptide sequence, a peptide variant thereof, or a non-peptide mimetic of such a sequence.

An aptamer includes a DNA or RNA molecule that binds to nucleic acids and proteins. An aptamer that binds to a protein (or binding domain) or a PTM Polynucleotide can be produced using conventional techniques, without undue experimentation. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

Antibodies for use in the present invention include but are not limited to synthetic antibodies, monoclonal antibodies, polyclonal antibodies, recombinant antibodies, antibody fragments (such as Fab, Fab', F(ab')2), dAb (domain antibody; see Ward, et al, 1989, Nature, 341:544-546), antibody heavy chains, intrabodies, humanized antibodies, human antibodies, antibody light chains, single chain Fᵥₛ (scFv) (e.g., including monospecific, bispecific etc.), anti-idiotypic (ant-Id) antibodies, proteins comprising an antibody portion, chimeric antibodies (for example, antibodies which contain the binding specificity of murine antibodies, but in which the remaining portions are of human origin), derivatives, such as enzyme conjugates or labeled derivatives, diabodies, linear antibodies, disulfide-linked Fvs (sdFv), multispecific antibodies (e.g., bispecific antibodies), epitope-binding fragments of any of the above, and any other modified configuration of an immunoglobulin molecule that comprises an antigen recognition site of the required specificity. An antibody includes an antibody of any type (e.g. IgA, IgD, IgE, IgG, IgM and IgY), any class (e.g. IgG1, IgG2, IgG3, IgG4, IgA1 and IgA2), or any subclass (e.g. IgG2a and IgG2b), and the antibody need not be of any particular type, class or subclass. An antibody may be from any animal origin including birds and mammals (e.g. human, murine, donkey, sheep, rabbit, goat, guinea pig, camel, horse, or chicken).

A “recombinant antibody” includes antibodies that are prepared, expressed, created or isolated by recombinant means, such as antibodies expressed using a recombinant expression vector transfected into a host cell, antibodies isolated from recombinant, combinatorial antibody libraries, antibodies isolated from an animal (e.g. a mouse or cow) that is transgenic and/or transchromosomal for human immunoglobin genes, or antibodies prepared, expressed, created or isolated by any other means that involves slicing of immunoglobulin gene sequences to other DNA sequences.

A “monoclonal antibody” refers to an antibody obtained from a population of homogenous or substantially homogenous antibodies. Generally each monoclonal antibody recognizes a single epitope on an antigen. In aspects of the invention, a monoclonal antibody is an antibody produced by a single hybridoma or other cell, and it specifically binds to only a PTM Polypeptide as determined, for example by ELISA or other antigen-binding or competitive binding assay known in the art. The term is not limited to a particular method for making the antibody and for example they may be produced by the hybridoma method or isolated from phage libraries using methods known in the art.

Antibodies, including monoclonal and polyclonal antibodies, fragments and chimeras, may be prepared using methods well known to those skilled in the art. Isolated native or recombinant polypeptides may be utilized to prepare antibodies. See, for example, Kohler et al. (1975) Nature 256:495-497; Kozbor et al. (1985) J. Immunol Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. Antibodies specific for polypeptide markers may also be obtained from scientific or commercial sources.

The term “detect” or “detecting” includes assaying, imaging or otherwise establishing the presence or absence of target markers, subunits thereof, or combinations of reagent bound targets, and the like, or assaying for, imaging, ascertaining, establishing, or otherwise determining one or more factual characteristics of preterm birth or similar conditions. The term encompasses diagnostic, prognostic, and monitoring applications for the PTM Polypeptides and PTM Polynucleotides.

“Microarray” and “array,” refer to nucleic acid or nucleotide arrays or protein or peptide arrays that can be used to detect biomolecules associated with spontaneous preterm birth, for instance to measure gene expression. A variety of arrays are made in research and manufacturing facilities worldwide, some of which are available commercially. By way of example, spotted arrays and in situ synthesized arrays are two kinds of nucleic acid arrays that differ in the manner in which the nucleic acid materials are placed onto the array substrate. A widely used in situ synthesized oligonucleotide array is GeneChip™ made by Affymetrix, Inc. Oligonucleotide probes that are 20- or 25-base long can be synthesized in silico on the array substrate. These arrays can achieve high densities (e.g., more than 40,000 genes per cm²). Generally spotted arrays have lower densities, but the probes, typically partial cDNA molecules, are much longer than 20- or 25-mers. Examples of spotted cDNA arrays include LifeArray made by Incyte Genomics and DermArray made by IntegriDerm (or Invitrogen). Pre-synthesized and amplified cDNA sequences are attached to the substrate of spotted arrays. Protein and peptide arrays also are known [(see for example, Zhu et al., Science 293:2101 (2001)]. The preparation, use, and analysis of microarrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I 995) PCT application WO95/35505; Heller, R. A. et al. (1997) Proc. Natl. Acad. Sci. 94:2150-2155; and Heller, M. J. et al. (1997) U.S. Pat. No. 5,605,662).

“Preterm Marker Polynucleotide(s)” or “PTM Polynucleotide(s)”, refers to a polynucleotide associated with spontaneous preterm birth, and/or encoding PTM Polypeptides including native-sequence polypeptides, polypeptide variants including a portion of a polypeptide, an isoform, precursor, complex, a chimeric polypeptide, or modified forms and derivatives of the polypeptides. A PTM Polynucleotide can be a polynucleotide listed in Table 2, 3 or 4 or a fragment thereof. In particular aspects of the invention the PTM Polynucleotides comprise, are chosen from or consist of ZNF605, LRRC41, PCDHGA12, ABT1, THBS3, VNN1, LOC100128908, CST13P, EEF1D, RPH3A, TRBV6-6, PLEC, MIR601, ZNF16, MIR3691, LOC101927441, ACAP2, ZNF324, SH3PXD2B and TBX21. In particular aspects of the invention the PTM Polynucleotides comprise, are chosen from or consist of ZNF605, LRRC41, PCDHGA12, ABT1, THBS3 and VNN1. In particular aspects of the invention the PTM Polynucleotides comprise, are chosen from or consist of LOC100128908, CST13P, EEF1D, RPH3A, TRBV6-6, PLEC, MIR601, and ZNF16. In particular aspects of the invention the PTM Polynucleotides comprise, are chosen from or consist of LOC100128908, MIR3691, LOC101927441, CST13P, ACAP2, ZNF324, SH3PXD2B and TBX21.

PTM Polynucleotides include complementary nucleic acid sequences, and nucleic acids that are substantially identical to these sequences (e.g. at least about 45%, preferably 50%, 55%, 60%, 65%, 70%, 75%, 80%, 85%, 90%, 95%, 97%, 98%, or 99% sequence identity).

PTM Polynucleotides also include sequences that differ from a native sequence due to degeneracy in the genetic code. As one example, DNA sequence polymorphisms within the nucleotide sequence of a PTM Polynucleotide may result in silent mutations that do not affect the amino acid sequence. Variations in one or more nucleotides may exist among individuals within a population due to natural allelic variation. DNA sequence polymorphisms may also occur which lead to changes in the amino acid sequence of a polypeptide.

Polynucleotides also include nucleic acids that hybridize under stringent conditions, preferably high stringency conditions to a PTM Polynucleotide. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0×sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

PTM Polynucleotides also include truncated nucleic acids or nucleic acid fragments and variant forms of the nucleic acids that arise by alternative splicing of an mRNA corresponding to a DNA.

PTM Polynucleotide markers are intended to include DNA and RNA (e.g. mRNA) and can be either double stranded or single stranded. A polynucleotide may, but need not, include additional coding or non-coding sequences, or it may, but need not, be linked to other molecules and/or carrier or support materials. The polynucleotides for use in the methods of the invention may be of any length suitable for a particular method. In certain applications the term refers to antisense polynucleotides (e.g. mRNA or DNA strand in the reverse orientation to sense polynucleotide markers).

The term “Preterm Marker Polypeptide(s)” or “PTM Polypeptide(s)” includes a polypeptide marker associated with spontaneous preterm birth. The term includes native-sequence polypeptides, isoforms, chimeric polypeptides, complexes, all homologs, fragments, precursors, and modified forms and derivatives of the markers. A “PTM Polypeptide” includes a marker encoded by or expressed by a polynucleotide listed in Table 2, 3 and/or 4. In particular aspects of the invention the PTM Polypeptides comprise, are chosen from or consist of the polypeptides expressed or encoded by ZNF605, LRRC41, PCDHGA12, ABT1, THBS3, VNN1, LOC100128908, CST13P, EEF1D, RPH3A, TRBV6-6, PLEC, MIR601, ZNF16, MIR3691, LOC101927441, ACAP2, ZNF324, SH3PXD2B and/or TBX21.

A “native-sequence polypeptide” comprises a polypeptide having the same amino acid sequence of a polypeptide derived from nature. Such native-sequence polypeptides can be isolated from nature or can be produced by recombinant or synthetic means. In aspects of the invention, the native-sequence polypeptide is produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “polypeptide variant” means a polypeptide having at least about 70-80%, preferably at least about 85%, more preferably at least about 90%, most preferably at least about 95% amino acid sequence identity with a native-sequence polypeptide. Particular polypeptide variants have at least 70-80%, 85%, 90%, 95% amino acid sequence identity to the sequences of the proteins expressed or encoded by the polynucleotides identified in Table 2, 3 or 4. Such variants include, for instance, polypeptides wherein one or more amino acid residues are added to, or deleted from, the N- or C-terminus of the full-length or mature sequences of the polypeptide, including variants from other species, but excludes a native-sequence polypeptide.

The invention also includes polypeptides that are substantially identical to the sequences of a polypeptide encoded or expressed by a PTM Polynucleotide, in particular a polypeptide expressed or encoded by a polynucleotide listed in Table 2, 3 or 4, (e.g. at least about 45%, preferably 50%, 55%, 60%, 65%, 70%, 75%, 80%, 85%, 90%, 95%, 97%, 98%, or 99% sequence identity), and more particularly polypeptides that retain the immunogenic activity of the corresponding native-sequence polypeptide.

Percent sequence identity of two amino acid sequences, or of two nucleic acid sequences is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

An allelic variant may also be created by introducing substitutions, additions, or deletions into a polynucleotide encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed and the activity of the polypeptide may be determined.

Polypeptide variants include polypeptides comprising amino acid sequences sufficiently identical to or derived from the amino acid sequence of a native polypeptide which comprise fewer amino acids than the full length polypeptides. A portion of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A naturally occurring allelic variant may contain conservative amino acid substitutions from the native polypeptide sequence or it may contain a substitution of an amino acid from a corresponding position in a polypeptide homolog, for example, a murine or rat polypeptide.

PTM Polypeptides include chimeric or fusion proteins. A “chimeric protein” or “fusion protein” comprises all or part (preferably biologically active) of a polypeptide expressed or encoded by a PTM Polynucleotide operably linked to a heterologous polypeptide (i.e., a polypeptide other than a polypeptide expressed or encoded by a PTM Polynucleotide). Within the fusion protein, the term “operably linked” is intended to indicate that a polypeptide expressed or encoded by a PTM Polynucleotide and the heterologous polypeptide are fused in-frame to each other. The heterologous polypeptide can be fused to the N-terminus or C-terminus of a polypeptide expressed or encoded by a PTM Polynucleotide. An example of a fusion protein is a GST fusion protein in which a polypeptide expressed or encoded by a PTM Polynucleotide is fused to the C-terminus of GST sequences. Another example of a fusion protein is an immunoglobulin fusion protein in which all or part of a polypeptide expressed or encoded by a PTM Polynucleotide is fused to sequences derived from a member of the immunoglobulin protein family. Chimeric and fusion proteins can be produced by standard recombinant DNA techniques.

A modified form of a polypeptide referenced herein includes modified forms of the polypeptides and derivatives of the polypeptides, including but not limited to glycosylated, phosphorylated, acetylated, methylated or lapidated forms of the polypeptides.

PTM Polypeptides may be prepared by recombinant or synthetic methods, or isolated from a variety of sources, or by any combination of these and similar techniques.

“Preterm prelabour rupture of membranes (PPROM)” is defined as spontaneous rupture of membranes at <37 weeks without labour, onset of spontaneous labour occurred at least 60 min after PPROM and subsequent preterm delivery.

The terms “sample”, “biological sample”, and the like mean a material known or suspected of expressing or containing one or more PTM Polynucleotides, and/or one or more PTM Polypeptides. A test sample can be used directly as obtained from the source or following a pretreatment to modify the character of the sample. A sample can be derived from any biological source, such as tissues, extracts, or cell cultures, including cells, cell lysates, and physiological fluids, such as, for example, whole blood, plasma, serum, saliva, ocular lens fluid, cerebral spinal fluid, sputum, sweat, urine, milk, ascites fluid, synovial fluid, peritoneal fluid, and the like. A sample can be obtained from animals, preferably mammals, most preferably humans. A sample can be treated prior to use, such as preparing plasma from blood, diluting viscous fluids, and the like. Methods of treatment can involve filtration, distillation, extraction, concentration, inactivation of interfering components, the addition of reagents, and the like.

In embodiments of the invention the sample is blood. In embodiments, the sample comprises subpopulations of leukocytes, in particular macrophages or lymphocytes. In embodiments of the invention, the sample comprises blood cells, particularly maternal peripheral blood cells, more particularly mononuclear leukocytes. In one embodiment, the sample is peripheral white blood cells. In an embodiment of the invention the sample is decidual cells.

The samples that may be analyzed in accordance with the invention include polynucleotides from clinically relevant sources, preferably expressed RNA or a nucleic acid derived therefrom (cDNA or amplified RNA derived from cDNA that incorporates an RNA polymerase promoter). The target polynucleotides can comprise RNA, including, without limitation total cellular RNA, poly(A)⁺ messenger RNA (mRNA) or fraction thereof, cytoplasmic mRNA, or RNA transcribed from cDNA (i.e., cRNA; see, e.g., Linsley & Schelter, U.S. patent application Ser. No. 09/411,074, filed Oct. 4, 1999, or U.S. Pat. Nos. 5,545,522, 5,891,636, or 5,716,785). Methods for preparing total and poly(A)⁺ RNA are well known in the art, and are described generally, for example, in Sambrook et al., (1989, Molecular Cloning—A Laboratory Manual (2nd Ed.), Vols. 1-3, Cold Spring Harbor Laboratory, Cold Spring Harbor, N.Y.) and Ausubel et al, eds. (1994, Current Protocols in Moelcular Biology, vol. 2, Current Protocols Publishing, New York). RNA may be isolated from eukaryotic cells by procedures involving lysis of the cells and denaturation of the proteins contained in the cells. Additional steps may be utilized to remove DNA. Cell lysis may be achieved with a nonionic detergent, followed by microcentrifugation to remove the nuclei and hence the bulk of the cellular DNA. (See Chirgwin et al., 1979, Biochemistry 18:5294-5299). Poly(A)+RNA can be selected using oligo-dT cellulose (see Sambrook et al., 1989, Molecular Cloning—A Laboratory Manual (2nd Ed.), Vols. 1-3, Cold Spring Harbor Laboratory, Cold Spring Harbor, N.Y.). In the alternative, RNA can be separated from DNA by organic extraction, for example, with hot phenol or phenol/chloroform/isoamyl alcohol.

It may be desirable to enrich mRNA with respect to other cellular RNAs, such as transfer RNA (tRNA) and ribosomal RNA (rRNA). Most mRNAs contain a poly(A) tail at their 3′ end allowing them to be enriched by affinity chromatography, for example, using oligo(dT) or poly(U) coupled to a solid support, such as cellulose or Sephadex™ (see Ausubel et al., eds., 1994, Current Protocols in Molecular Biology, vol. 2, Current Protocols Publishing, New York). Bound poly(A)+mRNA may be eluted from the affinity column using 2 mM EDTA/0.1% SDS.

Target polynucleotides can be detectably labeled at one or more nucleotides using methods known in the art. The label is preferably uniformly incorporated along the length of the RNA, and more preferably, is carried out at a high degree of efficiency. The detectable label can be a luminescent label, fluorescent label, bio-luminescent label, chemi-luminescent label, radiolabel, and colorimetric label. In a particular embodiment, the label is a fluorescent label, such as a fluorescein, a phosphor, a rhodamine, or a polymethine dye derivative. Commercially available fluorescent labels include, for example, fluorescent phosphoramidites such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.).

Target polynucleotides from a patient sample can be labeled differentially from polynucleotides of a standard. The standard can comprise target polynucleotides from normal individuals (e.g., those not afflicted with or pre-disposed to preterm birth, term deliveries), in particular pooled from samples from normal individuals. The target polynucleotides can be derived from the same individual, but taken at different time points, and thus indicate the efficacy of a treatment by a change in expression of the markers, or lack thereof, during and after the course of treatment.

“Spontaneous preterm birth” or “SPTB” refers to birth at <37 weeks of gestation.

“Spontaneous preterm labour (SPTL)” is defined as spontaneous onset of labour <37 weeks of gestation resulting in preterm delivery.

The terms “subject”, “individual” or “patient” refer to a warm-blooded animal such as a mammal. In particular, the terms refer to a human. A subject, individual or patient may be afflicted with or suspected of having or being pre-disposed to spontaneous preterm birth. The present invention may be particularly useful for determining spontaneous preterm birth development potential in at-risk patients suffering from particular spontaneous preterm birth predisposing conditions. Spontaneous preterm birth predisposing conditions include without limitation a previous history of preterm birth, previous history of abortion, anaemia, uterine factors such as uterine volume increase, uterine anomalies, trauma and infection. In aspects of the invention the predisposing conditions are history of preterm birth and history of abortion. In other aspects of the invention the predisposing conditions are history of abortion and anaemia. In embodiments of the invention “history of PTB” refers to any previous premature delivery, any type i.e. spontaneous or induced or medically instigated. In embodiments of the invention “history of abortion” refers to any previous abortion, any type i.e. spontaneous or induced. In embodiments of the invention “anaemia” refers to <120 g/L of haemoglobin occurring anytime during a current pregnancy prior to sampling (e.g., prior to 27-33 weeks).

“Statistically different levels”, “significantly altered”, or “significant difference” in levels of markers in a patient sample compared to a control or standard (e.g. normal levels or levels in other samples from a patient) may represent levels that are higher or lower than the standard error of the detection assay. In particular embodiments, the levels may be 1.5, 2, 2.3, 2.5, 2.6, 3, 4, 5, or 6 times higher or lower than the control or standard.

The “status” of a marker refers to the presence, absence or extent/level of the marker or some physical, chemical or genetic characteristic of the marker. Such characteristics include without limitation, expression level, activity level, structure (sequence information), copy number, post-translational modification etc. The status of a marker may be directly or indirectly determined. In some embodiments status is determined by determining the level of a marker in the sample. The “level” of an element in a sample has its conventional meaning in the art, and includes quantitative determinations (e.g. mg/mL, fold change, etc.) and qualitative determinations (e.g. determining the presence or absence of a marker or determining whether the level of the marker is high, low or even present relative to a standard).

The term “abnormal status” means that a marker's status in a sample is different from a reference status for the marker. A reference status may be the status of the marker in samples from normal subjects (e.g., term deliveries), averaged samples from subjects with the condition or sample(s) from the same subject taken at different times. An abnormal status includes an elevated, decreased, present or absent marker(s). Determining the level of a marker in a sample may include determining the level of the marker in a sample and abnormal status could be either lower levels (low status), undetectable levels (negative status) or higher levels (including any amount over zero) (elevated status) compared to a standard. A subject may have an increased likelihood of preterm birth if the status of a marker in the subject's sample is correlated with the condition (e.g. a level of the marker is closer to a standard or reference or is present in levels that exceed some threshold value where exceeding that value is correlated with the condition). A subject with an increased likelihood of preterm birth includes a subject with an abnormal status for a marker and as such the subject has a higher likelihood of preterm birth than if the subject did not have that status.

“Term delivery” is birth at ≧37 weeks of gestation irrespective of spontaneous onset or induction, vaginal delivery or caesarean section.

### Marker Sets

The invention provides a set of markers useful for detection, diagnosis, prevention and therapy of preterm birth. In particular, the invention provides gene marker sets that distinguish preterm birth and uses of such markers. In an aspect, the invention provides a method for classifying a sample as preterm birth comprising detecting a difference in the expression of a first plurality of genes relative to a control, the first plurality of genes consisting of at least 4, 5, 10, 15 or all of the genes corresponding to the markers listed in Table 2, 3 and/or 4. In one aspect, the genes correspond to markers for spontaneous preterm birth. In certain aspects, the genes comprise, are chosen from or consist of ZNF605, LRRC41, PCDHGA12, ABT1, THBS3, VNN1, LOC100128908, CST13P, EEF1D, RPH3A, TRBV6-6, PLEC, MIR601, ZNF16, MIR3691, LOC101927441, ACAP2, ZNF324, SH3PXD2B, and TBX21. In certain aspects, the genes comprise, are chosen from or consist of ZNF605, LRRC41, PCDHGA12, ABT1, THBS3 and VNN1. In certain aspects, the genes comprise, are chosen from or consist of LOC100128908, CST13P, EEF1D, RPH3A, TRBV6-6, PLEC, MIR601, and ZNF16. In certain aspects, the genes comprise, are chosen from or consist of LOC100128908, MIR3691, LOC101927441, CST13P, ACAP2, ZNF324, SH3PXD2B and TBX21.

Any of the markers provided herein may be used alone or with other markers of preterm birth or labor (e.g. fibronectin or phosphorylated insulin-like growth factor binding protein-1), or with markers for other phenotypes or conditions.

### Detection Methods

A variety of methods can be employed for the detection, diagnosis, monitoring, and prognosis of preterm birth, onset of preterm birth, or status of preterm birth involving one or more PTM Polypeptides and/or PTM Polynucleotides, and for the identification of subjects with a predisposition to preterm birth. Such methods may, for example, utilize PTM Polynucleotides, and fragments thereof, and binding agents (e.g. antibodies) against one or more PTM Polypeptides, including peptide fragments. In particular, the polynucleotides and antibodies may be used, for example, for (1) the detection of the presence of PTM Polynucleotide mutations, or the detection of either an over- or under-expression of PTM Polynucleotide mRNA relative to a non-preterm state, or the qualitative or quantitative detection of alternatively spliced forms of PTM Polynucleotide transcripts which may correlate with certain conditions or susceptibility toward spontaneous preterm birth; and (2) the detection of either an over- or an under-abundance of one or more PTM Polypeptides relative to a non-preterm birth state or a different stage or type or the presence of a modified (e.g., less than full length) PTM Polypeptide which correlates with a spontaneous preterm birth state or a progression toward spontaneous preterm birth, or a particular type or stage of spontaneous preterm birth.

If the gene(s) represent surface antigens or secreted peptides, antibodies can be raised and standard ELISA's developed. In addition, novel automated RNA extraction can be utilized, followed by multiplex, real time RT-PCR. For example, the MagNA Pure LC & LightCycler system from Roche Diagnostic is capable of accurately quantifying RNA expression in cells within 90 minutes.

The invention contemplates a method for detecting or monitoring spontaneous preterm birth, the stage or type of spontaneous preterm birth or onset of preterm birth, comprising producing a profile of levels of one or more PTM Polypeptide and/or PTM Polynucleotides, and optionally other markers associated with spontaneous preterm birth in a sample from a patient, and comparing the profile with a reference to identify a profile for the patient indicative of spontaneous preterm birth, the stage or type of spontaneous preterm birth or the onset of preterm birth. In an embodiment, the profile is represented as a graph or matrix (e.g., a heat map).

The methods described herein may be used to evaluate the probability of the presence of spontaneous preterm birth, or onset of spontaneous preterm birth, for example, in a sample freshly removed from a host. Such methods can be used to detect spontaneous preterm birth and help in the diagnosis and prognosis of spontaneous preterm birth. The methods can be used to detect the potential for spontaneous preterm birth and to monitor spontaneous preterm birth or a therapy.

The methods described herein can be adapted for diagnosing and monitoring preterm birth by detecting one or more PTM Polypeptides or PTM Polynucleotides in biological samples from a subject. These applications require that the amount of PTM Polypeptides or PTM Polynucleotides quantitated in a sample from a subject being tested be compared to a predetermined standard or cut-off value. The standard or cut-off value may correspond to levels quantitated for another sample or an earlier sample from the subject, or levels quantitated for a control sample. Levels for control samples from healthy subjects, different stages or types of spontaneous preterm birth, or term delivery subjects may be established by prospective and/or retrospective statistical studies. Healthy subjects who have no clinically evident preterm birth or abnormalities or have term deliveries may be selected for statistical studies. Diagnosis may be made by a finding of statistically different levels of detected PTM Polypeptides associated with preterm birth or PTM Polynucleotides, compared to a control sample or previous levels quantitated for the same subject.

The methods described herein may also use multiple markers for spontaneous preterm birth. Therefore, the invention contemplates a method for analyzing a biological sample for the presence of one or more PTM Polypeptides and PTM Polynucleotides, and other markers that are specific indicators of spontaneous preterm birth (e.g. fetal fibronectin or phosphorylated insulin-like growth factor binding protein-1). The methods described herein may be modified by including reagents to detect the additional markers.

The results of a subject's diagnosis, screening, prognosis or monitoring is typically displayed or provided to a user such as a clinician, health care worker or other caregiver, laboratory personnel or the patient. The results may be quantitative information (e.g. the level or amount of a marker compared to a control) or qualitative information (e.g. diagnosis of spontaneous preterm birth). The output can comprise guidelines or instructions for interpreting the results, for example, numerical or other limits that indicate the presence or absence of spontaneous preterm birth. The guidelines may also specify the diagnosis, for example whether there is a high risk of spontaneous preterm birth. The output can include tools for interpreting the results to arrive at a diagnosis, prognosis or treatment plan, for example, an output may include ranges or cut-offs for abnormal or normal status to arrive at a diagnosis, prognosis, or treatment plan. The output can also provide a recommended therapeutic plan, and it may include other clinical information and guidelines and instructions for interpreting the information.

Devices known in the art can be used to transmit the results of a method of the invention. Examples of output devices include without limitation, a visual output device (e.g. a computer screen or a printed paper), an auditory output device (e.g., a speaker), a printer or a patient s electronic medical record. The format of the output providing the results and related information may be a visual output (e.g., paper or a display on a screen), a diagram such as a graph, chart or voltammetric trace, an audible output (e.g. a speaker) or, a numerical value. In an aspect, the output is a numerical value, in particular the amount or relative amount of at least one marker in a subject's sample compared to a control. In an aspect, the output is a graph that indicates a value, such as an amount or relative amount, of the at least one marker in the sample from the subject on a standard curve. In an embodiment, the output (such as a graphical output) shows or provides a cut-off value or level that indicates the presence of high risk of spontaneous preterm birth. An output may be communicated to a user by physical, audible or electronic means, including mail, telephone, facsimile transmission, email or an electronic medical record.

### Nucleic Acid Methods/Assays

As noted herein, preterm birth or stage or type of same may be detected based on the level of PTM Polynucleotides in a sample. Techniques for detecting polynucleotides such as PCR and hybridization assays are well known in the art.

Probes may be used in hybridization techniques to detect polynucleotide markers. The technique generally involves contacting and incubating polynucleotides (e.g. recombinant DNA molecules, cloned genes) obtained from a sample from a patient or other cellular source with a probe under conditions favorable for the specific annealing of the probes to complementary sequences in the polynucleotides. After incubation, the non-annealed nucleic acids are removed, and the presence of polynucleotides that have hybridized to the probe if any are detected.

A “probe” to which a particular polynucleotide molecule specifically hybridizes according to the invention contains a complementary genomic polynucleotide sequence. The nucleotide sequences of the probes can be about 10-200 nucleotides in length. The probes can be genomic sequences of a species of organism, such that a plurality of different probes is present, with complementary sequences capable of hybridizing to the genome of such a species of organism. In other embodiments, the probes are about 10-30, 10-40, 20-50, 40-80, 50-150, 80-120 nucleotides in length, and in particular about 60 nucleotides in length.

The probes may comprise DNA or DNA mimics (e.g., derivatives and analogues) corresponding to a portion of an organism's genome, or complementary RNA or RNA mimics. Mimics are polymers comprising subunits capable of specific, Watson-Crick-like hybridization with DNA, or of specific hybridization with RNA. The nucleic acids can be modified at the base moiety, at the sugar moiety, or at the phosphate backbone. DNA can be obtained using standard methods such as PCR amplification of genomic DNA or cloned sequences. (See, for example, in Innis et al., eds., 1990, PCR Protocols: A Guide to Methods and Applications, Academic Press Inc., San Diego, Calif.). Computer programs known in the art can be used to design primers with the required specificity and optimal amplification properties, such as Oligo version 5.0 (National Biosciences). Controlled robotic systems may be useful for isolating and amplifying nucleic acids.

A nucleotide probe may be labeled with a detectable substance such as a radioactive label that provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect PTM Polynucleotides in human samples, e.g. peripheral blood leukocytes. The nucleotide probes may also be useful in the diagnosis of spontaneous preterm birth involving one or more PTM Polynucleotides, in monitoring the progression of pregnancy that results in spontaneous preterm birth, or monitoring a therapeutic treatment. In one embodiment, the nucleotide probes are associated with or covalently joined to a detectable label.

The levels of mRNA or polynucleotides derived therefrom can be determined using hybridization methods known in the art. Particular examples of methods based on hybridization analysis include, without limitation, northern blotting, RNA expression assays such as microarray analysis and in situ hybridization (Parker & Barnes, Methods in Molecular Biology 106: 247-283, 1999; RNAse protection assays (Hod, Biotechniques 13: 852-854, 1992); PCR based methods such as quantitative PCR, reverse transcription PCR (RT-PCR) (Weis et al., Trends in Genetics, 8:263-264, 1992), real-time reverse-transcription PCR (qRT-PCR), and in situ PCR. Methods to profile gene expression may also employ antibodies that can recognize sequence-specific duplexes such as RNA duplexes and DNA-RNA hybrid duplexes. Examples of methods based on sequencing include without limitation, serial analysis of gene expression (SAGE), deep sequencing (Creighton et al., Brief Bioinform. 10(5):490-2009) and gene expression analysis by massively parallel signature sequencing (MPSS).

In an example of a hybridization method, RNA can be isolated from a sample and separated on a gel. The separated RNA can then be transferred to a solid support and nucleic acid probes representing one or more markers can be hybridized to the solid support and the amount of marker-derived RNA is determined. Such determination can be visual, or machine-aided (e.g. use of a densitometer). Dot-blot or slot-blot may also be used to determine RNA. RNA or nucleic acids derived therefrom from a sample are labeled, and then hybridized to a solid support containing oligonucleotides derived from one or more marker genes that are placed on the solid support at discrete, easily-identifiable locations. Hybridization, or the lack thereof, of the labeled RNA to the solid support oligonucleotides is determined visually or by densitometer.

The detection of PTM Polynucleotides may involve the amplification of specific gene sequences using an amplification method such as polymerase chain reaction (PCR), followed by the analysis of the amplified molecules using techniques known to those skilled in the art. Suitable primers can be routinely designed by one of skill in the art. By way of example, at least two oligonucleotide primers may be employed in a PCR based assay to amplify a portion of a PTM Polynucleotide(s) derived from a sample, wherein at least one of the oligonucleotide primers is specific for (i.e. hybridizes to) a PTM Polynucleotide. The amplified cDNA is then separated and detected using techniques well known in the art, such as gel electrophoresis.

In order to maximize hybridization under assay conditions, primers and probes employed in the methods of the invention generally have at least about 60%, preferably at least about 75%, and more preferably at least about 90% identity to a portion of a PTM Polynucleotide; that is, they are at least 10 nucleotides, and preferably at least 20 nucleotides in length. In an embodiment the primers and probes are at least about 10-40 nucleotides in length.

Hybridization and amplification techniques described herein may be used to assay qualitative and quantitative aspects of PTM Polynucleotide expression. For example, RNA may be isolated from a cell type or tissue known to express a PTM Polynucleotide and tested utilizing the hybridization (e.g. standard Northern analyses) or PCR techniques referred to herein. The primers and probes may be used in the above-described methods in situ i.e. directly on tissue sections (fixed and/or frozen) of patient tissue.

In an aspect of the invention, a method is provided employing reverse transcriptase-polymerase chain reaction (RT-PCR), in which PCR is applied in combination with reverse transcription. Generally, RNA is extracted from a sample using standard techniques (for example, guanidine isothiocyanate extraction as described by Chomcynski and Sacchi, Anal. Biochem. 162:156-159, 1987) and is reverse transcribed to produce cDNA. The cDNA is used as a template for a polymerase chain reaction. The cDNA is hybridized to a set of primers, at least one of which is specifically designed against a PTM Polynucleotide sequence. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

Amplification may be performed on samples obtained from a subject with suspected spontaneous preterm birth and an individual who is not predisposed to spontaneous preterm birth or a term delivery individual. The reaction may be performed on several dilutions of cDNA spanning at least two orders of magnitude. A significant difference in expression in several dilutions of the subject sample as compared to the same dilutions of the control sample may be considered positive for the presence of spontaneous preterm birth.

In an embodiment, the invention provides methods for determining the presence or absence of spontaneous preterm birth in a subject comprising (a) contacting a sample obtained from the subject with oligonucleotides that hybridize to one or more PTM Polynucleotides; and (b) detecting in the sample a level of nucleic acids that hybridize to the polynucleotides relative to a predetermined cut-off value, and therefrom determining the presence or absence of spontaneous preterm birth in the subject. The subject may be symptomatic or asymptomatic, preferably asymptomatic.

The invention provides a method wherein a PTM Polynucleotide which is mRNA is detected by (a) isolating mRNA from a sample and combining the mRNA with reagents to convert it to cDNA; (b) treating the converted cDNA with amplification reaction reagents and nucleic acid primers that hybridize to one or more PTM Polynucleotides, to produce amplification products; (c) analyzing the amplification products to detect amounts of mRNA encoding PTM Polynucleotides; and (d) comparing the amount of mRNA to an amount detected against a panel of expected values for control subjects derived using similar nucleic acid primers.

PTM Polypeptide-positive samples or alternatively higher levels in patients compared to a control (e.g. normal tissue) may be indicative of preterm birth and/or that the patient is not responsive to or tolerant of a therapy. Alternatively, negative samples or lower levels compared to a control (e.g. normal samples or negative samples) may also be indicative of preterm birth.

In another embodiment, the invention provides methods for determining the presence or absence or risk of preterm birth in a subject comprising (a) contacting a sample obtained from the subject with oligonucleotides that hybridize to one or more PTM Polynucleotides; and (b) detecting in the sample levels of polynucleotides that hybridize to the PTM Polynucleotides relative to a predetermined cut-off value, and therefrom determining the presence or absence of preterm birth in the subject.

In embodiments, the PTM Polynucleotides encode one or more polynucleotides listed in Table 2, 3, or 4. In certain embodiments, the PTM Polynucleotides comprise, are chosen from or consist of ZNF605, LRRC41, PCDHGA12, ABT1, THBS3, VNN1, LOC100128908, CST13P, EEF1D, RPH3A, TRBV6-6, PLEC, MIR601, ZNF16, MIR3691, LOC101927441, ACAP2, ZNF324, SH3PXD2B, and TBX21. In certain embodiments, the PTM Polynucleotides comprise, are chosen from or consist of ZNF605, LRRC41, PCDHGA12, ABT1, THBS3 and VNN1. In certain embodiments, the PTM Polynucleotides comprise, are chosen from or consist of LOC100128908, CST13P, EEF1D, RPH3A, TRBV6-6, PLEC, MIR601, and ZNF16. In certain embodiments, the PTM Polynucleotides comprise, are chosen from or consist of LOC100128908, MIR3691, LOC101927441, CST13P, ACAP2, ZNF324, SH3PXD2B and TBX21.

Oligonucleotides or longer fragments derived from PTM Polynucleotides may be used as targets in a microarray as described herein. The microarray can be used to simultaneously monitor the expression levels of large numbers of genes. The microarray can also be used to identify genetic variants, mutations, and polymorphisms. The information from the microarray may be used to determine gene function, to understand the genetic basis of spontaneous preterm birth, to diagnose spontaneous preterm birth, and to develop and monitor the activities of therapeutic agents. The array can be used to assay expression of PTM Polynucleotides in the array. The invention also allows the quantitation of expression of one or more PTM Polynucleotides.

Thus, the invention also includes an array comprising one or more PTM Polynucleotides, in particular the markers listed in Table 2, 3 and/or 4, preferably the markers in Table 3, and optionally other markers. In certain embodiments, the array comprises the up-regulated polynucleotides in Table 2 or 4. In certain embodiments, the array comprises the down-regulated polynucleotides in Table 2 or 4. In certain embodiments, the array comprises ZNF605, LRRC41, PCDHGA12, ABT1, THBS3, VNN1, LOC100128908, CST13P, EEF1D, RPH3A, TRBV6-6, PLEC, MIR601, ZNF16, MIR3691, LOC101927441, ACAP2, ZNF324, SH3PXD2B, and TBX21. In certain embodiments, the array comprises ZNF605, LRRC41, PCDHGA12, ABT1, THBS3 and VNN1. In certain embodiments, the array comprises LOC100128908, CST13P, EEF1D, RPH3A, TRBV6-6, PLEC, MIR601, and ZNF16. In certain embodiments, the array comprises LOC100128908, MIR3691, LOC101927441, CST13P, ACAP2, ZNF324, SH3PXD2B and TBX21.

Microarrays typically contain, at separate sites, nanomolar quantities of individual genes, cDNAs, or ESTs on a substrate (e.g., nitrocellulose or silicon plate), or photolithographically prepared glass substrate. The arrays are hybridized to cDNA probes using conventional techniques with gene-specific primer mixes. The target polynucleotides to be analyzed are isolated, amplified and labeled, typically with fluorescent labels, radiolabels or phosphorous label probes. After hybridization is completed, the array is inserted into the scanner, where patterns of hybridization are detected. Data are collected as light emitted from the labels incorporated into the target, which becomes bound to the probe array. Probes that completely match the target generally produce stronger signals than those that have mismatches. The sequence and position of each probe on the array are known, and thus by complementarity, the identity of the target nucleic acid applied to the probe array can be determined.

Microarrays are prepared by selecting polynucleotide probes and immobilizing them to a solid support or surface. The probes may comprise DNA sequences, RNA sequences, copolymer sequences of DNA and RNA, DNA and/or RNA analogues, or combinations thereof. The probe sequences may be full or partial fragments of genomic DNA, or they may be synthetic oligonucleotide sequences synthesized either enzymatically in vivo, enzymatically in vitro (e.g., by PCR), or non-enzymatically in vitro.

The probe or probes used in the methods of the invention can be immobilized to a solid support or surface which may be either porous (e.g. gel), or non-porous. For example, the probes can be attached to a nitrocellulose or nylon membrane or filter covalently at either the 3′ or the 5′ end of the polynucleotide probe. The solid support may be a glass or plastic surface. In an aspect of the invention hybridization levels are measured to microarrays of probes consisting of a solid support on the surface of which are immobilized a population of polynucleotides.

In accordance with embodiments of the invention, a microarray is provided comprising a support or surface with an ordered array of hybridization sites or “probes” each representing one of the markers described herein. The microarrays can be addressable arrays, and in particular positionally addressable arrays. Each probe of the array is typically located at a known, predetermined position on the solid support such that the identity of each probe can be determined from its position in the array. In preferred embodiments, each probe is covalently attached to the solid support at a single site.

Microarrays used in the present invention are preferably (a) reproducible, allowing multiple copies of a given array to be produced and easily compared with each other; (b) made from materials that are stable under hybridization conditions; (c) small, (e.g., between 1 cm² and 25 cm², between 12 cm² and 13 cm², or 3 cm²; and (d) comprise a unique set of binding sites that will specifically hybridize to the product of a single gene in a cell (e.g., to a specific mRNA, or to a specific cDNA derived therefrom). However, it will be appreciated that larger arrays may be used particularly in screening arrays, and other related or similar sequences will cross hybridize to a given binding site.

In accordance with an aspect of the invention, the microarray is an array in which each position represents one of the markers described herein. Each position of the array can comprise a DNA or DNA analogue based on genomic DNA to which a particular RNA or cDNA transcribed from a genetic marker can specifically hybridize. A DNA or DNA analogue can be a synthetic oligomer or a gene fragment. In an embodiment, probes representing each of the PTM Polypeptides and PTM Polynucleotides are present on the array. In an embodiment, probes representing at least 5, 10, 15, or all of the PTM Polynucleotides of Table 2, 3 or 4 are present on the array. In an embodiment, probes representing the up-regulated polynucleotides listed in Table 2 or 4 are present on the array. In an embodiment, probes representing the down-regulated polynucleotides in Table 2 or 4 are present on the array. In an embodiment, probes representing ZNF605, LRRC41, PCDHGA12, ABT1, THBS3, VNN1, LOC100128908, CST13P, EEF1D, RPH3A, TRBV6-6, PLEC, MIR601, ZNF16, MIR3691, LOC101927441, ACAP2, ZNF324, SH3PXD2B, and TBX21 are present on the array. In an embodiment, probes representing ZNF605, LRRC41, PCDHGA12, ABT1, THBS3 and VNN1 are on the array. In an embodiment, probes representing LOC100128908, CST13P, EEF1D, RPH3A, TRBV6-6, PLEC, MIR601, and ZNF16 are on the array. In an embodiment, probes representing LOC100128908, MIR3691, LOC101927441, CST13P, ACAP2, ZNF324, SH3PXD2B and TBX21 are on the array.

Probes for the microarray can be synthesized using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between about 10 and about 500 bases, 20-100 bases, or 40-70 bases in length. Synthetic nucleic acid probes can include non-natural bases, such as, without limitation, inosine. Nucleic acid analogues such as peptide nucleic acid may be used as binding sites for hybridization. (see, e.g., Egholm et al., 1993, Nature 363:566-568; U.S. Pat. No. 5,539,083). Probes can be selected using an algorithm that takes into account binding energies, base composition, sequence complexity, cross-hybridization binding energies, and secondary structure (see Friend et al., International Patent Publication WO 01/05935, published Jan. 25, 2001). Positive control probes, (e.g., probes known to be complementary and hybridize to sequences in the target polynucleotides), and negative control probes, (e.g., probes known to not be complementary and hybridize to sequences in the target polynucleotides) are typically included on the array. Positive controls can be synthesized along the perimeter of the array or synthesized in diagonal stripes across the array. A reverse complement for each probe can be next to the position of the probe to serve as a negative control. The probes can be attached to a solid support or surface, which may be made from glass, plastic (e.g., polypropylene, nylon), polyacrylamide, nitrocellulose, gel, or other porous or nonporous material. The probes can be printed on surfaces such as glass plates (see Schena et al., 1995, Science 270:467-470). This method may be particularly useful for preparing microarrays of cDNA (See also, DeRisi et al., 1996, Nature Genetics 14:457-460; Shalon et al., 1996, Genome Res. 6:639-645; and Schena et al., 1995, Proc. Natl. Acad. Sci. U.S.A. 93:10539-11286).

High-density oligonucleotide arrays containing oligonucleotides complementary to defined sequences, at defined locations on a surface can be produced using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). Using these methods oligonucleotides (e.g., 60-mers) of known sequence are synthesized directly on a surface such as a derivatized glass slide. The array produced may be redundant, with several oligonucleotide molecules per RNA.

Microarrays can be made by other methods including masking (Maskos and Southern, 1992, Nuc. Acids. Res. 20:1679-1684). In an embodiment, microarrays of the present invention are produced by synthesizing polynucleotide probes on a support wherein the nucleotide probes are attached to the support covalently at either the 3′ or the 5′ end of the polynucleotide.

The invention provides microarrays comprising a disclosed marker set. In one embodiment, the invention provides a microarray for distinguishing preterm samples comprising a positionally-addressable array of polynucleotide probes bound to a support, the polynucleotide probes comprising a plurality of polynucleotide probes of different nucleotide sequences, each of the different nucleotide sequences comprising a sequence complementary and hybridizable to a plurality of genes, the plurality consisting of at least 5, 10, 15, or 20 of the genes corresponding to the markers listed in Table 2, 3, and/or 4. In embodiments, the microarray comprises or consists of the each of the embodiments of the plurality of genes disclosed herein.

In an aspect, the invention provides a method for classifying a sample as spontaneous preterm birth comprising detecting using a microarray a difference in the expression of a first plurality of genes relative to a control, the first plurality of genes consisting of at least 5, 10, 15, or 20, of the genes corresponding to the markers listed in Table 2, 3 or 4.

Arrays are also useful for ascertaining differential expression patterns of PTM Polynucleotides as described herein, and optionally other markers, in normal and abnormal samples. This may provide a battery of nucleic acids that could serve as molecular targets for diagnosis or therapeutic intervention.

### Protein Methods

Binding agents may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of preterm birth or stage or type of preterm birth in a subject may be determined by (a) contacting a sample from the subject with a binding agent; (b) detecting in the sample a level of PTM polypeptide(s) that binds to the binding agent; and (c) comparing the level of PTM Polypeptide(s) with a predetermined standard or cut-off value.

In particular embodiments of the invention, the binding agent is an antibody. Antibodies specifically reactive with one or more PTM Polypeptide, or derivatives, such as enzyme conjugates or labeled derivatives, may be used to detect one or more PTM Polypeptide in various samples (e.g. biological materials). They may be used as diagnostic or prognostic reagents and they may be used to detect abnormalities in the level of expression of one or more PTM Polypeptide, or abnormalities in the structure, and/or temporal, tissue, cellular, or subcellular location of one or more PTM Polypeptide. Antibodies may also be used to screen potentially therapeutic compounds in vitro to determine their effects on preterm birth involving one or more PTM Polypeptides, and other conditions. In vitro immunoassays may also be used to assess or monitor the efficacy of particular therapies.

In an aspect, the invention provides a diagnostic method for monitoring or diagnosing spontaneous preterm birth in a subject by quantitating one or more PTM Polypeptides in a biological sample from the subject comprising reacting the sample with antibodies specific for one or more PTM Polypeptides, which are directly or indirectly labeled with detectable substances and detecting the detectable substances. In a particular embodiment of the invention, PTM Polypeptides are quantitated or measured.

In an aspect of the invention, a method for detecting spontaneous preterm birth is provided comprising:
(a) obtaining a sample suspected of containing one or more PTM Polypeptides associated with spontaneous preterm birth;
(b) contacting said sample with antibodies that specifically bind to the PTM Polypeptides under conditions effective to bind the antibodies and form complexes;
(c) measuring the amount of PTM Polypeptides present in the sample by quantitating the amount of the complexes; and
(d) comparing the amount of PTM Polypeptides present in the samples with the amount of PTM Polypeptides in a control, wherein a change or significant difference in the amount of PTM Polypeptides in the sample compared with the amount in the control is indicative of spontaneous preterm birth.

In an embodiment, the invention contemplates a method for monitoring the progression of pregnancy that results in preterm birth in an individual, comprising:
(a) contacting antibodies which bind to one or more PTM Polypeptides with a sample from the individual so as to form complexes comprising the antibodies and one or more PTM Polypeptides in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of preterm birth in said individual.

The amount of complexes may also be compared to a value representative of the amount of the complexes from an individual not at risk of, or afflicted with, spontaneous preterm birth, or has had a term delivery at a different stage. A significant difference in complex formation may be indicative of spontaneous preterm birth, or an unfavorable prognosis.

In an embodiment of methods of the invention, the PTM Polypeptides encoded by the polynucleotides in Table 2, 3 or 4 are detected. In an embodiment of methods of the invention, PTM Polypeptides encoded by the polynucleotides comprising, chosen from, or selected from the up-regulated polynucleotides in Table 2 or 4 are detected in samples and increased levels, in particular significantly increased levels compared to a control is indicative of preterm birth. In a particular embodiment of methods of the invention, PTM Polypeptides encoded by the polynucleotides comprising, chosen from or selected from ZNF605, LRRC41, PCDHGA12, ABT1, THBS3, VNN1, LOC100128908, CST13P, EEF1D, RPH3A, TRBV6-6, PLEC, MIR601, ZNF16, MIR3691, LOC101927441, ACAP2, ZNF324, SH3PXD2B, and TBX21 are detected in samples and significantly different levels compared to a control (e.g., normal) is indicative of preterm birth. In a particular embodiment of methods of the invention, PTM Polypeptides encoded by the polynucleotides comprising, chosen from or selected from ZNF605, LRRC41, PCDHGA12, ABT1, THBS3 and VNN1 are detected in samples and significantly different levels compared to a control (e.g., normal) is indicative of preterm birth. In a particular embodiment of methods of the invention, PTM Polypeptides encoded by the polynucleotides comprising, chosen from or selected from LOC100128908, CST13P, EEF1D, RPH3A, TRBV6-6, PLEC, MIR601, and ZNF16 are detected in samples and significantly different levels compared to a control (e.g., normal) is indicative of preterm birth. In a particular embodiment of methods of the invention, PTM Polypeptides encoded by the polynucleotides comprising, chosen from or selected from LOC100128908, MIR3691, LOC101927441, CST13P, ACAP2, ZNF324, SH3PXD2B and TBX21 are detected in samples and significantly different levels compared to a control (e.g., normal) is indicative of preterm birth.

A particular embodiment of the invention comprises the following steps
(a) incubating a biological sample with first antibodies specific for one or more PTM Polypeptides which are directly or indirectly labeled with a detectable substance, and second antibodies specific for one or more PTM Polypeptides which are immobilized;
(b) detecting the detectable substance thereby quantitating PTM Polypeptides in the biological sample; and
(c) comparing the quantitated PTM Polypeptides with levels for a predetermined standard.

The standard may correspond to levels quantitated for samples from control subjects without spontaneous preterm birth (term delivery subjects) or from other samples of the subject. In an embodiment, increased levels of PTM Polypeptides as compared to the standard may be indicative of spontaneous preterm birth. In another embodiment, lower levels of PTM Polypeptides as compared to the standard may be indicative of spontaneous preterm birth.

Embodiments of the methods of the invention involve (a) reacting a biological sample from a subject with antibodies specific for one or more PTM Polypeptides which are directly or indirectly labelled with an enzyme; (b) adding a substrate for the enzyme wherein the substrate is selected so that the substrate, or a reaction product of the enzyme and substrate forms fluorescent complexes; (c) quantitating one or more PTM Polypeptides in the sample by measuring fluorescence of the fluorescent complexes; and (d) comparing the quantitated levels to levels obtained for other samples from the subject patient, or control subjects.

In another embodiment, the quantitated levels are compared to levels quantitated for control subjects (e.g. normal) wherein an increase in PTM Polypeptide levels compared with the control subjects is indicative of spontaneous preterm birth.

In further embodiment, the quantitated levels are compared to levels quantitated for control subjects (e.g. normal) wherein a decrease in PTM Polypeptide levels compared with the control subjects is indicative of spontaneous preterm birth.

Antibodies may be used in any known immunoassays that rely on the binding interaction between antigenic determinants of one or more PTM Polypeptide and the antibodies. Immunoassay procedures for in vitro detection of antigens in fluid samples are also well known in the art. [See for example, Paterson et al., Int. J. Can. 37:659 (1986) and Burchell et al., Int. J. Can. 34:763 (1984) for a general description of immunoassay procedures]. Qualitative and/or quantitative determinations of one or more PTM Polypeptide in a sample may be accomplished by competitive or non-competitive immunoassay procedures in either a direct or indirect format. Detection of one or more PTM Polypeptide using antibodies can be done utilizing immunoassays which are run in either the forward, reverse or simultaneous modes. Examples of immunoassays are radioimmunoassays (RIA), enzyme immunoassays (e.g. ELISA), immunofluorescence, immunoprecipitation, latex agglutination, hemagglutination, histochemical tests, and sandwich (immunometric) assays. These terms are well understood by those skilled in the art. A person skilled in the art will know, or can readily discern, other immunoassay formats without undue experimentation.

In an embodiment of the invention, an immunoassay for detecting more than one PTM Polypeptide in a biological sample comprises contacting binding agents that specifically bind to PTM Polypeptides in the sample under conditions that allow the formation of first complexes comprising a binding agent and PTM Polypeptides and determining the presence or amount of the complexes as a measure of the amount of PTM Polypeptides contained in the sample. In a particular embodiment, the binding agents are labeled differently or are capable of binding to different labels.

Binding agents (e.g. antibodies) may be used in immunohistochemical analyses, for example, at the cellular and sub-subcellular level, to detect one or more PTM Polypeptides, to localize them to particular cells and tissues, and to specific subcellular locations, and to quantitate the level of expression. Immunohistochemical methods for the detection of antigens in tissue samples are well known in the art. For example, immunohistochemical methods are described in Taylor, Arch. Pathol. Lab. Med. 102:112 (1978). Briefly, in the context of the present invention, a tissue sample obtained from a subject suspected of having spontaneous preterm birth is contacted with antibodies, preferably monoclonal antibodies recognizing one or more PTM Polypeptides. The site at which the antibodies are bound is determined by selective staining of the sample by standard immunohistochemical procedures. The same procedure may be repeated on the same sample using other antibodies that recognize one or more PTM Polypeptides. Alternatively, a sample may be contacted with antibodies against one or more PTM Polypeptides simultaneously, provided that the antibodies are labeled differently or are able to bind to a different label.

Binding agents, in particular antibodies, specific for one or more PTM Polypeptide may be labeled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol, enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

One of the ways a binding agent such as an antibody can be detectably labeled is to link it directly to an enzyme. The enzyme when later exposed to its substrate will produce a product that can be detected. Examples of detectable substances that are enzymes are horseradish peroxidase, beta-galactosidase, luciferase, alkaline phosphatase, acetylcholinesterase, malate dehydrogenase, ribonuclease, urease, catalase, glucose-6-phosphate, staphylococcal nuclease, delta-5-steriod isomerase, yeast alcohol dehydrogenase, alpha-glycerophosphate, triose phosphate isomerase, asparaginase, glucose oxidase, and acetylcholine esterase.

A bioluminescent compound may also be used as a detectable substance. Bioluminescence is a type of chemiluminescence found in biological systems where a catalytic protein increases the efficiency of the chemiluminescent reaction. The presence of a bioluminescent molecule is determined by detecting the presence of luminescence. Examples of bioluminescent detectable substances are luciferin, luciferase and aequorin.

Indirect methods may also be employed in which a primary antigen-antibody reaction is amplified by the introduction of a second antibody, having specificity for the antibody reactive against one or more PTM Polypeptides. By way of example, if the antibody having specificity against one or more PTM Polypeptides is a rabbit IgG antibody, the second antibody may be goat anti-rabbit gamma-globulin labelled with a detectable substance as described herein.

Methods for conjugating or labelling the binding agents such as antibodies discussed above may be readily accomplished by one of ordinary skill in the art. (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,”Anal. Biochem. 171:1-32, 1988 re methods for conjugating or labelling the antibodies with enzyme or ligand binding partner).

In the context of the methods of the invention, the sample, binding agents (e.g. antibodies specific for one or more PTM Polypeptides), or one or more PTM Polypeptides may be immobilized on a carrier or support. Examples of suitable carriers or supports are agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). Thus, the carrier may be in the shape of, for example, a tube, test plate, well, beads, disc, sphere, etc. The immobilized antibody may be prepared by reacting the material with a suitable insoluble carrier using known chemical or physical methods, for example, cyanogen bromide coupling. An antibody may be indirectly immobilized using a second antibody specific for the antibody. For example, mouse antibody specific for a PTM Polypeptide may be immobilized using sheep anti-mouse IgG Fc fragment specific antibody coated on the carrier or support.

Where a radioactive label is used as a detectable substance, one or more PTM Polypeptide may be localized by radioautography. The results of radioautography may be quantitated by determining the density of particles in the radioautographs by various optical methods, or by counting the grains.

One or more PTM Polypeptide antibodies may also be indirectly labeled with an enzyme using ligand binding pairs. For example, the antibodies may be conjugated to one partner of a ligand binding pair, and the enzyme may be coupled to the other partner of the ligand binding pair. Representative examples include avidin-biotin, and riboflavin-riboflavin binding protein. In an embodiment, the antibodies are biotinylated, and the enzyme is coupled to streptavidin. In another embodiment, an antibody specific for PTM Polypeptide antibody is labeled with an enzyme.

The invention relates to a method of characterizing or classifying a biological sample by detecting or quantitating in the sample one or more PTM Polypeptides extracted from the sample characteristic of spontaneous preterm birth, the method comprising assaying for differential expression of the PTM polypeptides in the sample by mass spectroscopy of proteins extracted from the sample. In an embodiment, differential expression of PTM Polypeptides is carried out using mass spectroscopy, in particular label-free SWATH (sequential window acquisition of all theoretical fragment-ion spectra) performed using a triple quadrupole mass spectrometer, or multiple reaction monitoring (MRM) using triple quadrupole mass spectrometer.

In an embodiment, the invention provides a method of characterizing or classifying a biological sample by detecting or quantitating in the sample one or more PTM Polypeptides extracted from the sample characteristic of spontaneous preterm birth comprising:
(a) extracting polypeptides from the sample and producing a profile of the polypeptides by subjecting the polypeptides to mass spectrometry; and
(b) identifying PTM Polypeptides by comparing the profile with a profile of PTM Polypeptides from a standard or control (e.g. a normal sample).

Step (b) may include using a statistical method to calculate a significance value for each of the markers in the profile.

Mass spectrometers that may be used to analyze the samples include a Matrix-Assisted Laser Desorption/Ioniation Time-of-Flight Mass Spectrometer (“MALDI-TOF”) (e.g. from PerSeptive Biosystems, Framingham, Mass.), an Electrospray Ionization (“ESI”) ion trap spectrometer, (e.g. from Finnigan MAT, San Jose, Calif.), an ESI quadrupole mass spectrometer (e.g. from Finnigan or Perkin-Elmer Corporation, Foster City, Calif.), a triple quadrupole/TOF mass spectrometer (ABSCIEX, Concord, Ontario), or a Surface Enhanced Laser Desorption/Ionization (SELDI-TOF) Mass Spectrometer (e.g. from Ciphergen Biosystems Inc.).

### Computer Systems

The analytic methods described herein can be implemented by use of computer systems and methods described below and known in the art. Thus the invention provides computer readable media comprising one or more PTM Polypeptides, and/or PTM Polynucleotides, and optionally other markers (e.g. markers of preterm birth). “Computer readable media” refers to any medium that can be read and accessed directly by a computer. Thus, the invention contemplates computer readable medium having recorded thereon markers identified for patients and controls. “Recorded” refers to a process for storing information on computer readable medium. The skilled artisan can readily adopt any of the presently known methods for recording information on computer readable medium to generate manufactures comprising information on one or more PTM Polypeptides, and/or PTM Polynucleotides, and optionally other markers.

A variety of data processor programs and formats can be used to store information on one or more PTM Polypeptides, and/or PTM Polynucleotides, and other markers on computer readable medium. Any number of dataprocessor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention also provides in an electronic system and/or in a network, a method for determining whether a subject has spontaneous preterm birth or is at risk of spontaneous preterm birth, comprising determining the presence or absence of one or more PTM Polypeptides, and/or PTM Polynucleotides, and optionally other markers, and based on the presence or absence of the one or more PTM Polypeptides, and/or PTM Polynucleotides, and optionally other markers, determining whether the subject has a pre-disposition to spontaneous preterm birth and optionally recommending a procedure or treatment.

The invention further provides in a network, a method for determining whether a subject has a pre-disposition to spontaneous preterm birth comprising: (a) receiving phenotypic and/or clinical information on the subject and information on one or more PTM Polypeptides, and/or PTM Polynucleotides, and optionally other markers associated with samples from the subject; (b) acquiring information from the network corresponding to the one or more PTM Polypeptides, and/or PTM Polynucleotides, and optionally other markers; and (c) based on the phenotypic information and information on the one or more PTM Polypeptides, and/or PTM Polynucleotides, and optionally other markers, determining whether the subject has a pre-disposition to spontaneous preterm birth; and (d) optionally recommending a procedure or treatment.

The invention still further provides a system for identifying selected records that identify spontaneous preterm birth. A system of the invention generally comprises a computer; a database server coupled to the computer; a database coupled to the database server having data stored therein, the data comprising records of data comprising one or more PTM Polypeptides, and/or PTM Polynucleotides, and optionally other markers, and a code mechanism for applying queries based upon a desired selection criteria to the data file in the database to produce reports of records which match the desired selection criteria.

In an aspect of the invention a method is provided for detecting cells or tissues associated with spontaneous preterm birth using a computer having a processor, memory, display, and input/output devices, the method comprising the steps of:
(a) creating records of one or more PTM Polypeptides, and/or PTM Polynucleotides, and optionally other markers, identified in a sample suspected of containing PTM Polypeptides, and/or PTM Polynucleotides associated with spontaneous preterm birth;
(b) providing a database comprising records of data comprising one or more PTM Polypeptides, and/or PTM Polynucleotides, and optionally other markers of spontaneous preterm birth; and
(c) using a code mechanism for applying queries based upon a desired selection criteria to the data file in the database to produce reports of records of step (a) which provide a match of the desired selection criteria of the database of step (b) the presence of a match being a positive indication that the markers of step (a) have been isolated from cells or tissue that are associated with spontaneous preterm birth.

The invention contemplates a business method for determining whether a subject has a pre-disposition to spontaneous preterm birth comprising: (a) receiving phenotypic and/or clinical information on the subject and information on one or more PTM Polypeptides, and/or PTM Polynucleotides, and optionally other markers, associated with samples from the subject; (b) acquiring information from a network corresponding to one or more PTM Polypeptides, and/or PTM Polynucleotides, and optionally other markers; and (c) based on the phenotypic information, information on one or more PTM Polypeptides, and/or PTM Polynucleotides encoding the markers, and optionally other markers, and acquired information, determining whether the subject has a pre-disposition to spontaneous preterm birth; and (d) optionally recommending a procedure or treatment.

In an aspect of the invention, the computer systems, components, and methods described herein are used to monitor preterm birth or determine the stage or type of spontaneous preterm birth. The computer systems, components and methods may also include clinical variables, in particular history of PTB, history of abortion, consumption of alcohol, antepartum haemorrhage in first and/or second trimester, presence of Group B streptococcus, urinary tract infection and anaemia, more particularly history of PTB, history of abortion and anaemia.

### Screening Methods

The invention also contemplates methods for evaluating test agents or compounds for their ability to prevent, inhibit or reduce spontaneous preterm birth, potentially contribute to spontaneous preterm birth, or inhibit or enhance a type of spontaneous preterm birth. Test agents and compounds include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. The agents or compounds may be endogenous physiological compounds or natural or synthetic compounds.

The invention provides a method for assessing the potential efficacy of a test agent for inhibiting spontaneous preterm birth or onset of spontaneous preterm birth in a patient, the method comprising comparing:
(a) levels of one or more PTM Polypeptides, and/or PTM Polynucleotides, and optionally other markers in a first sample obtained from a patient and exposed to the test agent; and
(b) levels of one or more PTM Polypeptides and/or PTM Polynucleotides, and optionally other markers in a second sample obtained from the patient, wherein the sample is not exposed to the test agent, wherein a significant difference in the levels of expression of one or more PTM Polypeptides, and/or PTM Polynucleotides, and optionally the other markers, in the first sample, relative to the second sample, is an indication that the test agent is potentially efficacious for inhibiting spontaneous preterm birth or onset of spontaneous preterm birth in the patient.

The first and second samples may be portions of a single sample obtained from a patient or portions of pooled samples obtained from a patient.

In an aspect, the invention provides a method of selecting an agent for inhibiting preterm birth or onset of spontaneous preterm birth in a patient comprising:
(a) obtaining a sample from the patient;
(b) separately maintaining aliquots of the sample in the presence of a plurality of test agents;
(c) comparing one or more PTM Polypeptides, and/or PTM Polynucleotides, and optionally other markers, in each of the aliquots; and
(d) selecting one of the test agents which alters the levels of one or more PTM Polypeptides, and/or PTM Polynucleotides, and optionally other markers in the aliquot containing that test agent, relative to other test agents.

Still another aspect of the present invention provides a method of conducting a drug discovery business comprising:
(a) providing one or more methods or assay systems of the invention for identifying agents that inhibit, prevent or reduce spontaneous preterm birth, onset of spontaneous preterm birth, or affect a stage or type of spontaneous preterm birth in a patient;
(b) conducting therapeutic profiling of agents identified in step (a), or further analogues thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The invention also contemplates a method of assessing the potential of a test compound to contribute to spontaneous preterm birth comprising:
(a) maintaining separate aliquots of body fluid, cells or tissues from a patient with preterm labour in the presence and absence of the test compound; and
(b) comparing one or more PTM Polypeptides, and/or PTM Polynucleotides, and optionally other markers in each of the aliquots.

A significant difference between the levels of the markers in the aliquot maintained in the presence of (or exposed to) the test compound relative to the aliquot maintained in the absence of the test compound, indicates that the test compound possesses the potential to contribute to spontaneous preterm birth.

### Kits

The invention also contemplates kits for carrying out the methods of the invention. Kits may typically comprise two or more components required for performing a diagnostic assay. Components include but are not limited to compounds, reagents, containers, and/or equipment.

The methods described herein may be performed by utilizing pre-packaged diagnostic kits comprising one or more specific PTM Polypeptide, PTM Polynucleotide, binding agent (e.g. antibody), probe or primer described herein, which may be conveniently used, e.g., in clinical settings to screen and diagnose patients and to screen and identify those individuals exhibiting a predisposition to preterm birth.

In an aspect, a container with a kit comprises a binding agent as described herein. By way of example, the kit may contain antibodies or antibody fragments which bind specifically to epitopes of one or more PTM Polypeptides, and optionally other markers, antibodies against the antibodies labeled with an enzyme, and a substrate for the enzyme. In an embodiment of the invention, the kit includes antibodies or fragments of antibodies which bind specifically to an epitope of one or more polypeptides expressed or encoded by polynucleotides listed in Table 2, 3 and/or 4. The kit may also contain microtiter plate wells, standards, assay diluent, wash buffer, adhesive plate covers, and/or instructions for carrying out a method of the invention using the kit.

In an aspect, a kit is provided comprising at least one oligonucleotide probe or primer, as described herein, that hybridizes to a PTM Polynucleotide. Such an oligonucleotide may be used, for example, within a PCR or hybridization procedure. In an embodiment of the invention, the kit comprises oligonucleotide probes or primers that hybridize to one or more polynucleotides listed in Table 2, 3 and/or 4.

In an aspect, the invention provides a kit containing a microarray described herein ready for hybridization to target PTM Polynucleotides, plus software for the data analysis of the results. The software to be included with the kit comprises data analysis methods, in particular mathematical routines for marker discovery, including the calculation of correlation coefficients between clinical categories and marker expression. The software may also include mathematical routines for calculating the correlation between sample marker expression and control marker expression, using array-generated fluorescence data, to determine the clinical classification of the sample.

The reagents suitable for applying the screening methods of the invention to evaluate compounds may be packaged into convenient kits described herein providing the necessary materials packaged into suitable containers. The invention relates to a kit for assessing the suitability of each of a plurality of test compounds for inhibiting preterm birth in a patient. The kit comprises reagents for assessing one or more PTM Polypeptides or PTM Polynucleotides, and optionally a plurality of test agents or compounds. The invention contemplates a kit for assessing the presence of cells and tissues associated with preterm birth wherein the kit comprises antibodies specific for one or more PTM Polypeptides, or primers or probes for PTM Polynucleotides, and optionally probes, primers or antibodies specific for other markers associated with preterm birth or labor (e.g. fetal fibronectin). Additionally the invention provides a kit for assessing the potential of a test compound to contribute to spontaneous preterm birth. The kit comprises cells and tissues associated with preterm birth and reagents for assessing one or more PTM Polypeptides, PTM Polynucleotides, and optionally other markers associated with spontaneous preterm birth.

The following non-limiting example is illustrative of the present invention:

### EXAMPLE

The following methods were used in the study described in this example.

### Patient Recruitment

The study population was drawn from a subset of women who participated in the All Our Babies (AOB) study, a community based longitudinal pregnancy cohort in Calgary, Alberta, Canada approved by the Conjoint Health Research Ethics Board, University of Calgary (Ethics #20821 and #22128). Pregnant women receiving prenatal viral serology testing were recruited through a partnership with Calgary Laboratory Service between May 2008 and December 2010. Written consent was obtained at the time of the first blood collection. Women also completed a survey about lifestyle, psychosocial and health care utilisation (prenatal care, social support, symptoms of stress, anxiety and depression, and breastfeeding) at <25 weeks, 34-36 weeks of gestation and 4 months postpartum [McDonald et al, 2013].

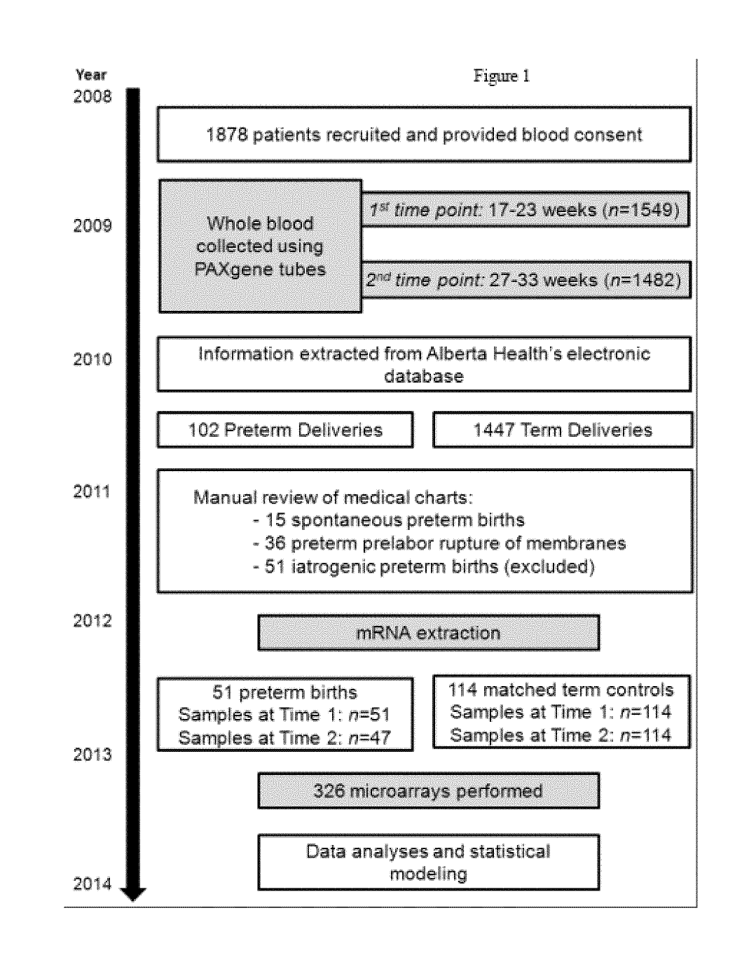

Detailed inclusion and exclusion criteria for the AOB study have been described [Gracie et al, 2010]. Briefly, inclusion criteria were ≧18 years of age, gestation age <18 weeks at time of recruitment and singleton pregnancy; exclusion criteria were multifetal pregnancy and pre-existing medical conditions (diabetes, high blood pressure, autoimmune disorders, kidney disease, cardiovascular disease or chronic infection). Clinical and antenatal records were extracted from the Alberta Health electronic database. Women who had PTB were confirmed by a manual review of the medical charts. Clinical data were unavailable for four women who delivered out of province at term. FIG. 1 summarises the patient recruitment, patient phenotyping and selection process.

Spontaneous preterm labour (SPTL) is defined as spontaneous onset of labour ≦37 weeks of gestation resulting in preterm delivery. Preterm prelabour rupture of membranes (PPROM) is defined as spontaneous rupture of membranes at <37 weeks without labour, onset of spontaneous labour occurred at least 60 min after PPROM and subsequent preterm delivery. Term delivery is birth at ≧37 weeks of gestation irrespective of spontaneous onset or induction, vaginal delivery or caesarean section. In Calgary, anaemia is defined as <120 g/L of haemoglobin; oligohydramnios and polyhydramnios are diagnosed using an amniotic fluid index of <5 cm and >20 cm, respectively. Antepartum haemorrhage is defined as recurrent haemorrhage at ≦20 or >20 weeks of gestation. Urinary tract infection (UTI) was indicated positive by either microscopic or macroscopic urinalysis, or culture. Demographic, clinical, labour and delivery variables were analysed using one-way ANOVA, Student's t-test, Chi-squared test or Fisher's exact test (R, version 3.2.1).

### Sample Collection and Processing

Maternal blood samples were collected at 17-23 (time point 1, T1) and 27-33 weeks of gestation (time point 2, T2) into four PAXgene™ blood RNA tubes (PreAnalytix/BD Canada, Mississauga, ON, Canada) and stored at −80° C. until analysis.

### RNA Extraction, Quality Check and Microarray

Total RNA was extracted using the PAXgene™ blood RNA Kit (PreAnalytix/QIAGEN, Toronto, ON, Canada) adhering to the manufacturer's protocol. All samples had RNA integrity number of >7 (RNA 6000 Nano Kit and Agilent 2100 BioAnalyzer; Agilent Technologies, Santa Clara, Calif.) and were hybridised to Affymetrix Human Gene 2.1 ST (Affymetrix, Santa Clara, Calif.). Microarray was performed by The Centre for Applied Genomics (TCAG; The Hospital for Sick Children, Toronto, ON, Canada). Data were deposited into the National Center for Biotechnology Information Gene Expression Omnibus (accession number: GSE59491; https//www.ncbi.nlm.nih.gov/geo/query/acc.cgi!acc=GSE59491).

### Differential Gene Expression Analyses

Microarray CEL files were normalised using Robust Multi-array Average (Bioconductor, R) [Irizarry et al, 2003], probes were annotated using Custom (Gene)Chip Definition Files for Entrez Gene (version 18) [Dai et al, 2005], gene expression lower than the 25th percentile were removed, and differential gene expression was analysed using limma [Smyth. 2004] with multiple hypothesis testing (false discovery rate, FDR). limma analyses were adjusted for gestational age at sampling, significant demographic or clinical variables when appropriate (see Items 1 to 6, Differential Gene Expression Analyses below). Differential gene expression was initially performed between SPTL and PPROM at T1 or T2 to determine if any gene was differentially expressed between these two subtypes of SPTB. There was no differentially expressed gene between SPTL and PPROM, thus, SPTL and PPROM were combined into a SPTB group for all subsequent analyses. Five limma analyses were conducted. The first two limma analyses determined genes differentially expressed between women who had SPTBs and term deliveries at (1) T1 or (2) T2.

Investigating the temporal gene expression from T1 to T2 provides information about the progression of pregnancies that result in normal term deliveries or SPTBs. Hence, the third and fourth limma analyses were performed to identify genes displaying temporal changes from T1 to T2 within women who had (3) SPTB or (4) term deliveries. The fifth analysis was conducted to identify (5) genes whose expression fold change from T1 to T2 were different between SPTBs and term deliveries. Genes with FDR<0.05 were selected for qRT-PCR validation.

### Differential Gene Expression Analyses

1. To compare between spontaneous preterm labour (SPTL) and preterm prelabour rupture of membranes (PPROM) at time point 1 (T1, 17-23 weeks) and at time point 2 (T2, 27-33 weeks)
   Gestational age, pre-pregnancy BMI, polyhydramnios and APH after 20 weeks were adjusted for in the limma analyses. No differentially expressed genes were obtained hence SPTL and PPROM were combined into a spontaneous preterm birth (SPTB) group.
2. To compare between SPTB and Term delivery at T1
   Gestational age, alcohol consumption, history of PTB, history of abortion and urinary tract infection (UTI) present before T1 were adjusted for in the limma analysis.
3. To compare between SPTB and Term delivery at T2
   Gestational age, alcohol consumption, history of PTB, history of abortion, UTI present before T2 and anaemia present before T2 were adjusted for in the limma analysis
4. To compare temporal gene expression between T2 and T1 in SPTB
   Women were accounted for in this paired limma analysis.
5. To compare temporal gene expression between T2 and T1 in Term
   Women were accounted for in this paired limma analysis.
6. To compare temporal gene expression between SPTB and Term
   Women were accounted for in this paired limma analysis

### Gene Set Enrichment

Pre-ranked Gene Set Enrichment Analyses [Subramanian et al, 2005] was utilised to determine significantly enriched gene sets/pathways (Gene Ontology Biological Processes, Reactome, KEGG and BioCarta, versions 5.1) between women who had SPTBs and term deliveries at (1) T1 or (2) T2; gene sets associated with temporal changes within women who had (3) SPTBs or (4) term deliveries, and (5) gene sets that reflect the difference in gene expression fold change between SPTB and term delivery.

### Qualitative Real Time PCR

Genes (limma FDR<0.05) that displayed >25% increase or >15% decrease, and CEL files with arbitrary intensity expression values of at least four were selected for qRT-PCR validation [Morey et al, 2006; Dallas et al, 2005; Heng et al, 2014]. Primers were designed using Primer BLAST; pooled cDNA (paired samples from six women) were used to determine primer specificity and efficiency; and primer efficiencies (90%-105%) were determined using five-point standard curves. qRT-PCR was carried out in quadruplicate, and quantification cycle (Cq) of all genes were <32. Gene expression was analysed using the 2(-Delta Delta Ct) method. Using CFX Manager 3.1 (BIO-RAD, Hercules, Calif.), qRT-PCR expression data were corrected for primer efficiencies and normalised to the geometric mean Cq of three optimised housekeeping genes (TBP, SDHA and YWHAZ [Heng et al, 2014]; average expression stability was M<0.5 [Vandesompele et al, 2002]) to obtain the first Delta Ct. Wilcoxon test was used to compare the relative gene expression between paired samples (second Delta Ct). Correlation between microarray and qRT-PCR was performed using Spearman's rho.

Multivariate Models Associated with Spontaneous Preterm Birth

Three multivariate models were constructed to identify gene expression at Ti (Model A), T2 (Model B), and gene expression fold change from T1 to T2 (Model C) associated with SPTB (Statistical Analysis System, version 9.3, SAS Institute Inc, Cary, N.C.). Clinical factors occurring before T1 or T2 that were significant in univariate analyses were entered into a multivariate logistic regression analysis. Clinical factors occurring before T1 that remained significant in the multivariate clinical factor analysis were included in Model A; and significant clinical factors occurring before T2 in the multivariate clinical factor analysis were included for Models B and C. Gestational age were also accounted for in the Models (see Items 1 to 3, Constructing Multivariate Associated with Spontaneous Preterm Birth below). To assess validity, each Model was subjected to ten five-fold cross-validation with gene selection occurring at every fold. To evaluate the importance and effect of adjusting gene expression with clinical factors, models were also built without clinical factors (i.e. using gene expression only; see Items 1 and 2 of Five-Fold Cross-Validation of Multivariate Models With and Without Clinical Factors below). The probability cut-off was 0.5, predictive performances such as area under receiver operator characteristic curve (ROC AUC) are the average of ten cross-validation runs. ROC AUCs were graphed using ROCR, R [Sing et al, 2005].

Constructing Multivariate Models Associated with Spontaneous Preterm Birth
1. Model A
   A. Performed limma and adjusted for gestational age at T1. Selected genes whose |t value|>3 (n=320).
   B. Performed univariate analysis (logistic regression) on each selected gene from A. by adjusting for history of preterm birth and history of abortion (multivariate analyses' significant clinical factors before T1). Chose top 20 genes for C (to avoid overfitting).
   C. Performed multivariate logistic regression (stepwise selection) using genes from B., and history of preterm birth, history of abortion and gestational age at T1 were fixed in the model.
2. Model B
   A. Performed limma and adjusted for gestational age at T2. Selected genes whose |t value|>3 (n=195).
   B. Performed univariate analysis (logistic regression) on each selected gene from A. by adjusting for history of abortion and anaemia (multivariate analyses' significant clinical factors before T2). Chose top 20 genes for C (to avoid overfitting).
   C. Performed multivariate logistic regression (stepwise selection) using genes from B., and history of abortion and anaemia and gestational age at T2 were fixed in the model.
3. Model C
   A. Performed limma and adjusted for gestational age at T1 and the number of weeks between T1 and T2. Extracted genes whose |t value|>3 (n=115).
   B. Performed univariate analysis (logistic regression) on each selected gene from A. by adjusting for history of abortion and anaemia (multivariate analyses' significant clinical factors before T2), and gestational age at T1. Chose top 20 genes for C (to avoid overfitting).
   C. Performed multivariate logistic regression (stepwise selection) using genes from B., and history of abortion, anaemia and gestational age at T1 were fixed in the model.
   Gestational age at T1 is used (instead of T2) in C as a way to “adjust” for the temporal gene expression obtained in A.
      Five-Fold Cross-Validation of Multivariate Models with and without Clinical Factors
1. Each Model was subjected to ten five-fold cross-validation with gene selection occurring at every fold, with clinical factors and gestational age fixed in the model. The selected genes at each fold were recorded.
2. To evaluate the importance and effect of adjusting gene expression with clinical factors, models were built using gene expression only. This was carried out by immediately repeating the training of the exact fold in 1. using the recorded genes and excluding the clinical factors.

The results of the study are described below.

After excluding iatrogenic PTB, there were 51 SPTB cases where 10 were extreme SPTB (<32 weeks) and four delivered before T2. The average time from PPROM until labour onset was 27.7 hours. Power calculations indicated that a control group of at least 85 term women was required to match 51 SPTB, with an effect size of 0.5, significance level of 0.05 and power of 0.8. Term delivery controls (n=114, power=0.84) were matched to SPTB cases drawn from baseline survey at <25 weeks of gestation by parity (no previous birth/at least one previous birth), maternal age (<35 years versus ≧35 years), pre-pregnancy body mass index (<18.5 kg/m², 18.5-24.9 kg/m², 25-29.9 kg/m², ≧30 kg/m²), ethnicity (Caucasian versus non-Caucasian), and pre-pregnancy smoking status (yes/no). A total of 326 microarrays (165 women) were performed. Eleven clinical variables were significantly associated with SPTB (Table 1).

Differential Gene Analysis Performed using Limma

There was no differentially expressed gene at FDR<0.05 but at FDR<0.10, there were 0 and 26 differentially expressed genes between women who had SPTB or term delivery at T1 and T2, respectively. There were 234 and 2329 genes that displayed significant temporal differences within women who had SPTBs or term deliveries, respectively (FDR<0.05). There was no gene expression fold change that was significantly different between SPTB and term delivery.

### Gene Set Enrichment

Significantly enriched gene sets (FDR<0.05) were identified in the study. At both sampling time points, gene sets and pathways associated with inflammation were upregulated in women with SPTBs compared to women who had term deliveries (n=37 upregulated gene sets at T1, n=103 at T2; 22 common gene sets). These inflammatory pathways include leukocyte migration, lysosomes, NF-kB activation, pathways involving cytokines and their receptors (e.g. IL1, IL2, IL6, IFN, IL1R, TNFR2, CCR3, CXCR4 and CD40) as well as toll-like and NOD-like receptor signalling. In contrast, women with SPTBs had lower RNA metabolism, RNA processing and T cell activation (including CTLA4 pathway) compared to women who had term deliveries (n=163 downregulated gene sets at T1, n=100 at T2; 77 common gene sets).

As pregnancy progressed from T1 to T2, women with SPTB demonstrated increased cellular proliferation, cell migration signalling pathway (by L1) and extracellular matrix degradation involving lysosomes (n=32 upregulated gene sets), and decreased cellular transcription (n=1 downregulated gene set). In women with term deliveries, there was increased signalling for cell migration, haemostasis, apoptosis and immune response (n=114 upregulated gene sets); while there was decreased lymphocyte activation and NCAM cell adhesive interactions as pregnancy progressed to T2 (n=36 downregulated gene sets). When investigating whether any gene set was enriched for genes whose expression fold change were different between SPTBs and term deliveries, there was no up-regulated gene set but “membrane fusion” (n=1) was significantly down-regulated in SPTB.

qRT-PCR Validation

Validation was performed on 192 samples randomly chosen from 48 women who had term deliveries (96 paired-samples) and 50 SPTBs (92 paired-samples, 4 single samples at T1). This resulted in using two 384-well plates to screen for each gene of interest. Significant temporal gene expression in women with SPTBs or term deliveries was subjected to validation (Table 2). Thirteen unique genes were successfully validated using qRT-PCR (p<0.05, Wilcoxon test). There was a significant correlation between microarray and qRT-PCR data (Spearman's rho=0.934, p<0.001).

Multivariate Models Associated with Spontaneous Preterm Birth Clinical Factors

Significant clinical variables determined after delivery (placental abruption, chorioamnionitis, gestational age at delivery and birth weight), during late gestation (Group B streptococcus) or those that did not achieve significance before T2 were not considered. Significant clinical factors with events occurring before T1 were alcohol consumption, history of PTB, history of abortion and UTI before T1. History of PTB (p=0.0024) and history of abortion (p=0.0025) remained significant in the multivariate analysis and were included in Model A. Alcohol consumption, history of PTB, history of abortion, UTI before T2 and anaemia before T2 were significant clinical factors with events occurring before T2; history of abortion (p=0.0002) and anaemia before T2 (p=0.0003) remained significant in the multivariate analysis and were included in Models B and C.

### Multivariate Gene Expression Models

After adjusting for gestational age and clinical factors that remained significant after multivariate analyses, candidate genes were incorporated into the multivariate logistic regression (stepwise selection) to build Models A, B and C (Table 3). As the prevalence of SPTB in this study was 31% (51 SPTB out of 165 total deliveries; higher than the average PTB rate of 10%), positive and negative predictive values, and false positive and negative rates must be interpreted with caution as these values are dependent on the prevalence of the disease, i.e. PTB in the study population, whilst sensitivity, specificity and ROC AUC are prevalence independent.

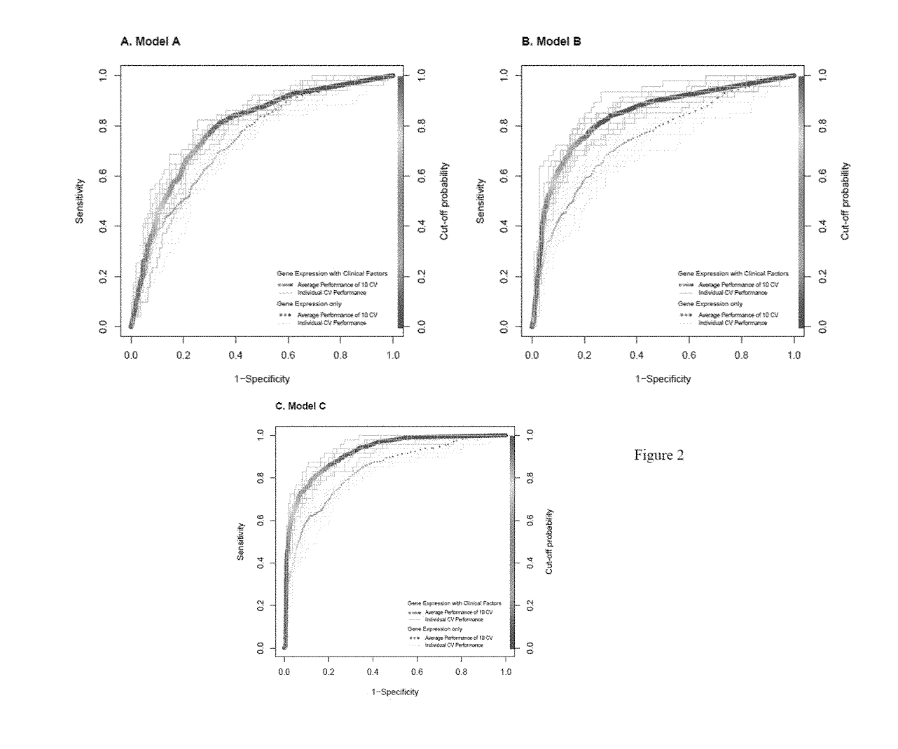

The ROC AUCs of Models A, B and C with clinical factors were 11.0%, 12.0% and 10.9% higher than the ROC AUCs of their corresponding Models without clinical factors (FIG. 2). This resulted in 18.3%, 34.8% and 23.0% increased sensitivity, and 3.4%, 0.9% and 4.7% increased specificity in Models A, B and C with clinical factors, respectively, when compared to Models without clinical factors. Models B and C were more sensitive than Model A (62.3% and 64.7% versus 52.4%), most likely due to the shorter time frame from sampling at T2 to SPTB (average of 4.7 weeks after T2).

The results of the study are discussed below.

This study profiled pregnant whole blood mRNA and investigated the association of whole blood gene expression with impending SPTB in asymptomatic women at two clinically relevant time points. T1 generally corresponds to when fetal anatomy ultrasound scan is performed and T2 is when blood is collected for gestational diabetes screening. This large, paired and unique dataset also provide glimpses of pregnancy progression that result in either SPTB or term delivery. The eleven clinical variables significantly associated with SPTB agree with previous reports [Kuhrt et al, 2016; Räisänen et a12013; Makhlouf et al, 2014; Gilbert et al, 2013; van den Broek et al, 2014; Baig et al, 2013; Yi et al, 2013]. Although the association of inflammation with the general physiology of labour at term or preterm gestation is well documented [Thomson et al, 1999; Heng et al, 2014; Enquobahrie et al, 2009; Osman et al, 2006], the paired data and gene set enrichment analyses show for the first time, that inflammation is consistently elevated at 17-23 and 23-33 weeks of gestation in the blood of asymptomatic women who had SPTBs compared to women with term deliveries. Lastly, the integration of clinical data alongside gene expression enhanced the sensitivity of the models to predict SPTB.

Gene set analyses provide biological knowledge of how genes interact and orchestrate pathways. Despite not observing any significant gene at FDR<0.05, numerous gene sets were significantly associated with SPTB. Circulating maternal leukocytes may pick up ‘signals’ from gestational tissues and respond by altering their gene expression. The most striking gene set enrichment result was that women with SPTB have increased interleukin signalling, mainly driven by IL1 and IL6, and leukocyte migration into gestational tissues as early as 18 weeks compared to women who had term deliveries. The early migration of leukocytes into the cervix may accelerate its ripening process and lead to SPTB [Word et al, 2007; Liggins, 1989]. The increased signalling of IL1 and IL6 can also contribute to SPTB by increasing in oxytocin and prostaglandin production leading to accelerated cervical ripening [Ulmsten et al, 1982; Watari et al, 1999; and Schmitz et al, 2003], early myometrial contractions [Friebe-Hoffmann et al, 2001; Kennard et al, 1995; Rauk et al. 2000] and premature fetal membranes rupture [Romero et al, 1989; Fortunato et al, 2003].

The AOB cohort is representative of the pregnant population in urban centres across Canada [McDonald et al, 2013; and Leung et al, 2013]. The SPTB rate in the province of Alberta is 6.2% [Public Health Agency of Canada, 2012]. 110 SPTBs were expected from 1878 AOB participants, but only 51 SPTBs were identified after manual chart review. Thus, the AOB population was not enriched with women at high risk of SPTB. Nevertheless, the predictive models developed in the AOB cohort may offer unique possibilities for research, clinical care and resource utilization. The key to preventing SPTB is the early identification of asymptomatic women at increased risk. The ability to identify these women can aid study groups to focus on ‘high risk’ women and avoid unnecessary (and expensive) research on those destined for term delivery when evaluating new interventions to prevent PTB. The development of a SPTB predictive tool will also allow further refinement of the subsets of women who will benefit from the existing preventive strategies of progesterone therapy [Meis et al, 2003; da Fonseca et al, 2003], cervical cerclage [Berghella et al, 2005; Simcox et al, 2009] or pessary [Goya et al, 2012].

Many research studies have investigated tools to identify “high-risk” asymptomatic women. For example, the absence of fFN in the cervicovaginal fluid is a classic negative predictor of PTB [Duhig et al, 2009; Abbott et al, 2015], especially for symptomatic women [Honest et al, 2002]. Dekker et al. reported average predictive capacity for SPTB and PPROM using clinical risk factors, cervical length and uterine artery Doppler ultrasound measurements at 19-21 weeks of gestation [Dekker et al, 2012]. They also reported a minimal overlap of risk factors for SPTB and PPROM, highlighting the heterogeneous condition of PTB. Kuhrt et al. recently developed a validated tool comprising of cervical length, fFN, history of SPTB/PPROM to predict “high-risk” asymptomatic women with ROC AUCs ranging from 0.77 to 0.99, sensitivity between 54.5% and75.0%, and specificity between 63.5% and 97.7% [Kuhrt et al, 2016]. The performances of the Models B and C described herein are comparable to Kuhrt et al. In addition, it might be more advantageous to screen for biomarkers in maternal blood as blood is easily accessible, minimally invasive and can be collected in most women as part of standard antenatal care [Heng et al, 2014; Heng et al, 2015]. This is in contrast to fFN screening where the test is limited to a subset of eligible women, e.g. had no prior vaginal/cervical examination, unprotected sexual intercourse and/or antepartum haemorrhage.

Models B and C are useful SPTB screening tools since most PTB occurs after 28 weeks of gestation [Martin et al, 2013]. The slight difference in predictive efficacies between Models B and C, and the simplicity of obtaining one sample at T2 makes Model B more practical clinically. It is important to note that although the predictive efficacies for the Models were reported using a 0.5 cut-off (Table 3), cut-off probability thresholds can be tailored for clinical use, e.g. a higher sensitivity test to predict SPTB (FIG. 2). Collectively, given the multiple aetiologies of SPTB, a set of diagnostic markers including biochemical, clinical variables, cervical length as well as whole blood gene expression should improve PTB prediction in asymptomatic women in the future.

In conclusion, this current work has shown that clinical factors and whole blood gene expression are associated with SPTB in asymptomatic women. Gene set enrichment analyses revealed elevated inflammation in women with SPTB. The ability to implement an effective screening test during antenatal care for SPTB would enable strategic and personalised antenatal care, to improve outcomes for infants and families.

In summary, the aim of the study described in this Example was to investigate maternal whole blood gene expression profiles associated with spontaneous preterm birth (SPTB, <37 weeks) in asymptomatic pregnant women. The study population was a matched subgroup of women (51 SPTBs, 114 term delivery controls) who participated in the All Our Babies (AOB) community based cohort in Calgary (n=1878). Maternal blood at 17-23 (time point 1, T1) and 27-33 weeks of gestation (T2) were collected. Total RNA was extracted and microarray was performed on 326 samples (165 women). Univariate analyses determined significant clinical factors and differential gene expression associated with SPTB. Thirteen genes were validated using qRT-PCR. Three multivariate logistic models were constructed to identify gene expression at T1 (Model A), T2 (Model B), and gene expression fold change from T1 to T2 (Model C) associated with SPTB. All models were adjusted for clinical factors. Model C can predict SPTB with 65% sensitivity and 88% specificity in asymptomatic women after adjusting for history of abortion and anaemia. Clinical data enhanced the sensitivity of the Models to predict SPTB. In conclusion, clinical factors and whole blood gene expression are associated with SPTB in asymptomatic women. An effective screening tool for SPTB during pregnancy would enable targeted preventive approaches and personalised antenatal care.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, websites, patents and patent applications referred to herein are incorporated by reference in their entirety to the same extent as if each individual publication, patent or patent application was specifically and individually indicated to be incorporated by reference in its entirety. All publications, websites, patents and patent applications mentioned herein are incorporated herein by reference for the purpose of describing and disclosing the domains, cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a cell” includes a plurality of such cells, reference to the “antibody” is a reference to one or more antibodies and equivalents thereof known to those skilled in the art, and so forth.

TABLE 1
Demographic, clinical, labour and delivery characteristics of the 165 participants.

| | | | | | SPTB |
|---|---|---|---|---|---|
| Spontaneous Preterm Birth (SPTB) | | | | SPTL vs | vs |
| | | SPTB | | PPROM | Term |
| | | (SPTL and | Term | vs Term | p- |
| SPTL | PPROM | PPROM) | Birth | p-value | value |

Patient Demographics

| | | | | | | |
|---|---|---|---|---|---|---|
| Women, n | 15 | 36 | 51 | 114 | | |
| Maternal age, mean | 31.1 ± 4.9 | 31.3 ± 4.6 | 31.2 ± 4.7 | 31.1 ± 4.7 | 0.910 | 0.850 |
| years ± SD | | | | | | |
| Pre-pregnancy BMI, | 21.9 ± 2.8 | 26.6 ± 9.1 | 25.3 ± 8.0 | 25.8 ± 72 | 0.321 | 0.702 |
| mean ± SD | | | | | | |
| Ethnicity | | | | | 0.559 | 0.946 |
| Caucasian, n (%) | 10 | 29 | 39 (76.5) | 85 (74.6) | | |
| Non-Caucasian, n (%) | 5 | 7 | 12 (23.5) | 29 (25.4) | | |
| Smoking during pregnancy | | | | | 0.379 | 0.367 |
| Yes, n (%) | 2 | 8 | 10 (19.6) | 14 (12.7) | | |
| No, n (%) | 13 | 28 | 41 (80.4) | 96 (87.3) | | |
| Consumption of alcohol | | | | | 0.021 | 0.038 |
| during pregnancy | | | | | | |
| Yes, n (%) | 3 | 4 | 7 (13.7) | 4 (3.6) | | |
| No, n (%) | 12 | 32 | 44 (86.3) | 106 (96.4) | | |

Clinical Characteristics

| | | | | | | |
|---|---|---|---|---|---|---|
| Gravidity, mean ± SD | 2.7 ± 1.7 | 2.0 ± 1.3 | 2.2 ± 1.4 | 2.0 ± 1.2 | 0.109 | 0.410 |
| Parity | | | | | 0.480 | 0.984 |
| Nulliparous, n (%) | 6 | 21 | 27 (52.9) | 60 (54.5) | | |
| Multiparous, n (%) | 9 | 15 | 24 (47.1) | 50 (45.5) | | |
| History of previous PTB | | | | | 0.001 | 0.001 |
| Previous PTB, n (%) | 4 | 7 | 11 (21.6) | 4 (3.6) | | |
| No previous PTB, n (%) | 11 | 29 | 40 (78.4) | 106 (96.4) | | |
| History of abortion | | | | | 0.002 | 0.001 |
| At least one abortion, n (%) | 5 | 9 | 14 (27.5) | 8 (7.3) | | |
| No previous abortion, n (%) | 10 | 27 | 37 (72.5) | 102 (92.7) | | |
| Mode of conception | | | | | 0.188 | 0.267 |
| Spontaneous | 13 | 34 | 47 (92.2) | 106 96.4) | | |
| conception, n (%) | | | | | | |
| Assisted reproductive | 2 | 2 | 4 (7.8) | 4 (3.6) | | |
| technologies, n (%) | | | | | | |
| Oligohydramnios | | | | | 0.800 | 1.00 |
| Present, n (%) | 0 | 2 | 2 (3.9) | 4 (3.6) | | |
| Absent, n (%) | 15 | 34 | 49 (96.1) | 106 (96.4) | | |
| Polyhydramnios | | | | | 0.002 | 0.094 |
| Present, n (%) | 3 | 0 | 3 (5.9) | 1 (0.9) | | |
| Absent, n (%) | 12 | 36 | 48 (94.1) | 109 (99.1) | | |
| Gestational diabetes during | | | | | 0.216 | 0.350 |
| pregnancy | | | | | | |
| Present, n (%) | 2 | 3 | 5 (9.8) | 5 (4.5) | | |
| Absent, n (%) | 13 | 33 | 46 (90.2) | 105 (95.5) | | |
| Antepartum haemorrhage | | | | | 0.004 | 0.009 |
| during pregnancy | | | | | | |
| ≧1 episode of bleeding, n (%) | 7 | 9 | 16 (31.4) | 14 (12.7) | | |
| None, n (%) | 8 | 27 | 35 (68.6) | 96 (87.3) | | |
| Antepartum haemorrhage | | | | | 0.419 | 0.353 |
| <20 weeks of gestation(i.e. | | | | | | |
| threatened miscarriage) | | | | | | |
| ≧1 episode, n (%) | 3 | 6 | 9 (17.6) | 12 (10.9) | | |
| None, n (%) | 12 | 30 | 42 (82.4) | 98 (89.1) | | |
| Antepartum haemorrhage | | | | | 0.021 | 0.262 |
| >20 weeks of gestation | | | | | | |
| ≧1 episode, n (%) | 6 | 4 | 10 (21.3) | 14 (12.7) | | |
| None, n (%) | 8 | 29 | 37 (78.7) | 96 (87.3) | | |
| Urinary tract infection | | | | | <0.001 | 0.001 |
| during pregnancy | | | | | | |
| Present, n (%) | 4 | 3 | 7 (14.0) | 1 (0.9) | | |
| Absent, n (%) | 11 | 32 | 43 (86.0) | 109 (99.1) | | |
| Urinary tract infection before | | | | | 0.029 | 0.029 |
| first sampling (17-23 weeks | | | | | | |
| of gestation) | | | | | | |
| Present, n (%) | 1 | 2 | 3 (6.0) | 0 (0.0) | | |
| Absent, n (%) | 14 | 33 | 47 (94.0) | 110 (100.0) | | |
| Urinary tract infection before | | | | | <0.001 | 0.003 |
| second sampling (27-33 | | | | | | |
| weeks of gestation) | | | | | | |
| Present, n (%) | 3 | 2 | 5 (10.0) | 0 (0.0) | | |
| Absent, n (%) | 12 | 33 | 45 (90.0) | 110 (100.0) | | |
| Anaemia during pregnancy | | | | | <0.001 | <0.001 |
| Anaemic, n (%) | 4 | 8 | 12 (23.5) | 3 (2.7) | | |
| Non-anaemic, n (%) | 11 | 28 | 39 (76.5) | 107 (97.3) | | |
| Anaemia before first | | | | | 0.099 | 0.099 |
| sampling (17-23 weeks of | | | | | | |
| gestation) | | | | | | |
| Present, n (%) | 0 | 2 | 2 (3.9) | 0 (0.0) | | |
| Absent, n (%) | 15 | 34 | 49 (96.1) | 110 (100.0) | | |
| Anaemia before second | | | | | <0.001 | <0.001 |
| sampling (27-33 weeks of | | | | | | |
| gestation) | | | | | | |
| Present, n (%) | 4 | 8 | 12 (23.5) | 1 (0.9) | | |
| Absent, n (%) | 11 | 28 | 39 (76.5) | 109 (99.1) | | |
| Group B Streptococcus in | | | | | 0.071 | 0.043 |
| vaginal tract (>36 weeks of | | | | | | |
| gestation) | | | | | | |
| Present, n (%) | 2 | 2 | 4 (7.8) | 24 (21.8) | | |
| Absent, n (%) | 13 | 34 | 47 (92.2) | 86 (78.2) | | |
| Placenta Praevia | | | | | 0.143 | 0.327 |
| Present, n (%) | 0 | 3 | 3 (5.9) | 2 (1.8) | | |
| Absent, n (%) | 15 | 33 | 48 (94.1) | 108 (98.2) | | |

Labour and Delivery Characteristics

| | | | | | | |
|---|---|---|---|---|---|---|
| Abruptio Placentae | | | | | 0.004 | 0.004 |
| Yes, n (%) | 1 | 5 | 6 (11.8) | 1 (0.9) | | |
| No, n (%) | 14 | 31 | 45 (88.2) | 109 (99.1) | | |
| Chorioamnionitis | | | | | 0.004 | 0.004 |
| Yes, n (%) | 1 | 5 | 6 (11.8) | 1 (0.9) | | |
| No, n (%) | 14 | 31 | 45 (88.2) | 109 (99.1) | | |
| Gestational age at delivery, | 33.5 ± 2.6 | 33.6 ± 2.6 | 33.6 ± 2.6 | 39.2 ± 1.2 | <0.001 | <0.001 |
| mean weeks ± SD | | | | | | |
| Birth weight, mean | 2257 ± 551 | 2363 ± 618 | 2332 ± 596 | 3384 ± 473 | <0.001 | <0.001 |
| grams ± SD | | | | | | |
| Neonatal Sex | | | | | 0.683 | 0.601 |
| Male, n (%) | 8 | 22 | 30 (58.8) | 71 (64.5) | | |
| Female, n (%) | 7 | 14 | 21 (41.2) | 39 (35.5) | | |

TABLE 2
Microarray and quantitative real time-PCR of 13 unique genes (ranked by fold change).

| | | Effi- | qRT-PCR | | Microarray | |
|---|---|---|---|---|---|---|
| Forward Primer | Reverse Primer | ciency | Fold | p- | Fold | |
| (5′ to 3′) | (5′ to 3′) | (%) | Change | value | Change | FDR |

Spontaneous Preterm Birth: Genes up-regulated at T2 compared to T1

| | | | | | | | |
|---|---|---|---|---|---|---|---|
| ABCA13 | GCCCTGCTGTGGAAG | AACAGGATACAAGGCC | 104.0 | 1.95 | <0.001 | 1.49 | <0.001 |
| | AATTG [SEQ ID NO: 1] | AGAAGA [SEQ ID NO: 2] | | | | | |
| MYOF | CTGGTGGGGAAGTGG | CCAAACGTTGGAACAA | 104.5 | 1.53 | >0.001 | 1.27 | >0.001 |
| | AAGATT [SEQ ID NO: 3] | AGCCT [SEQ ID NO: 4] | | | | | |
| SASH1 | CTGGAAGTGGAGAAA | GCTACAGAAGCCAAGC | 98.6 | 1.33 | >0.001 | 1.25 | >0.001 |
| | CCCGA [SEQ ID NO: 5] | GACT [SEQ ID NO: 6] | | | | | |
| LAP3 | ACAGGTGCCATGGAT | CTGTTTCAATGCTGGCC | 92.9 | 1.43 | >0.001 | 1.25 | >0.001 |
| | GTAGC [SEQ ID NO: 7] | TCG [SEQ ID NO: 8] | | | | | |

Spontaneous Preterm Birth: Genes down-regulated at T2 compared to T1

| | | | | | | | |
|---|---|---|---|---|---|---|---|
| FCER1A | CCTGCCATGGAATCC | TTCTGAGGGACTGCTAA | 98.1 | 0.66 | <0.001 | 0.68 | <0.001 |
| | CCTAC [SEQ ID NO: 9] | CACG [SEQ ID NO: 10] | | | | | |
| CPA3 | CCGCTACATCTATGG | CCCAGGTCATAAGCCCA | 96.4 | 0.69 | >0.001 | 0.70 | 0.001 |
| | CCCAAT [SEQ ID NO: 11] | GTC [SEQ ID NO: 12] | | | | | |
| ABCG1 | TGAGAAAGGACTCCT | ACCGAGTCCCTCATGAT | 90.0 | 0.64 | >0.001 | 0.78 | <0.001 |
| | CGTCCAT [SEQ ID NO: 13] | GCT [SEQ ID NO: 14] | | | | | |
| ABCA1 | AGCACAGGCTITGAC | GCTCGCAATFACGGGGT | 98.3 | 0.73 | 0.005 | 0.80 | 0.002 |
| | CGATA [SEQ ID NO: 15] | TTT [SEQ ID NO: 16] | | | | | |

Term Delivery: Genes up-regulated at T2 compared to T1

| | | | | | | | |
|---|---|---|---|---|---|---|---|
| OLFM4 | CAGCTGGAGGTGGAG | CCACGATTTCTCGGCG | 94.3 | 2.3 | >0.001 | 1.88 | <0.001 |
| | ATAAGAA [SEQ ID NO: 17] | AATG [SEQ ID NO: 18] | | | | | |
| DEFA3 | CTTGCTGCCATTCTCC | CATGTTTTTCCTTGAGC | 96.1 | 2.7 | <0.001 | 1.86 | <0.001 |
| | TGGT [SEQ ID NO: 19] | CTGGA [SEQ ID NO: 20] | | | | | |
| DEFA4 | TGCTCTTCAGGTTTCA | GCGTGCAGCAGTATGT | 98.6 | 2.8 | <0.001 | 1.82 | <0.001 |
| | GGCTC [SEQ ID NO: 21] | GAAA [SEQ ID NO: 22] | | | | | |
| CEACAM8 | TCGTGTCAACCCCAAA | ACAAAGAGTTGTGTTA | 92.2 | 2.4 | <0.001 | 1.80 | <0.001 |
| | TTTTTACG [SEQ ID NO: 23] | AAGATGCTG [SEQ ID NO: 24] | | | | | |

Term Delivery: Genes down-regulated at T2 compared to T1

| | | | | | | | |
|---|---|---|---|---|---|---|---|
| FCER1A | CCTGCCATGGAATCCC | TTCTGAGGGACTGCTA | 98.1 | 0.71 | <0.001 | 0.78 | <0.001 |
| | CTAC [SEQ ID NO: 25] | ACACG [SEQ ID NO: 26] | | | | | |
| CPA3 | CCGCTACATCTATGGC | CCCAGGTCATAAGCCC | 96.4 | 0.57 | <0.001 | 0.79 | <0.001 |
| | CCAAT [SEQ ID NO: 27] | AGTC [SEQ ID NO: 28] | | | | | |
| HDC | GTCAAAGTTGTGGTCG | TTAGCTCCGCCCTTCAA | 91.0 | 0.83 | 0.124 | 0.82 | <0.001 |
| | CTGT [SEQ ID NO: 29] | AGT [SEQ ID NO: 30] | | | | | |
| ABCG1 | TGAGAAAGGACTCCT | ACCGAGTCCCTCATGA | 90.0 | 0.81 | <0.001 | 0.83 | <0.001 |
| | CGTCCAT [SEQ ID NO: 31] | TGCT [SEQ ID NO: 32] | | | | | |

Housekeeping Genes

| | | | | |
|---|---|---|---|---|
| SDHA | TGGGAACAAGAGGGC | CCACCACTGCATCAAA | 99.5 | — |
| | ATCTG [SEQ ID NO: 33] | TTCATG [SEQ ID NO: 34] | | |
| TBP | TGCACAGGAGCCAAG | CACATCACAGCTCCCC | 95.8 | — |
| | AGTGAA [SEQ ID NO: 35] | ACCA [SEQ ID NO: 36] | | |
| YWHAZ | ACTTTTGGTACATTGT | CCGCCAGGACAAACCA | 91.0 | — |
| | GGCTTCAA [SEQ ID NO: 37] | GTAT [SEQ ID NO: 38] | | |

TABLE 3
Multivariate models (Models A, B and C) associated with spontaneous preterm birth (SPTB) at 17-23 (T1) and 27-33
(T2) weeks of gestation.

| Average of ten five-fold cross validations (cut-off = 0.5) | | | | | | |
|---|---|---|---|---|---|---|
| | | | Positive | Negative | False | False |
| | | | Predictive | Predictive | Positive | Negative |
| | Sensitivity | Specificity | Value* | Value* | Rate* | Rate* |
| ROC AUC | (%) | (%) | (%) | (%) | (%) | (%) |

SPTB models with gene expression and significant clinical factors included

| | | | | | | | | |
|---|---|---|---|---|---|---|---|---|
| A | ZNF605, LRRC41, PCDHGA12, ABT1, | 0.780 | 52.4 | 84.3 | 61.0 | 79.2 | 15.7 | 47.6 |
| | THBS3, VNN1, history of PTB and history | | | | | | | |
| | of abortion | | | | | | | |
| B | LOC100128908, CST13P, EEF1D, | 0.838 | 62.3 | 87.3 | 67.8 | 84.5 | 12.7 | 37.7 |
| | RPH3A, TRBV6-6, PLEC, MIR601, | | | | | | | |
| | ZNF16, history of abortion and anaemia* | | | | | | | |
| C | LOC100128908, MIR3691, | 0.841 | 64.7 | 88.3 | 70.1 | 85.4 | 11.7 | 35.3 |
| | LOC101927441, CST13P, ACAP2, | | | | | | | |
| | ZNF324, SH3PXD2B, TBX21, history of | | | | | | | |
| | abortion and anaemia | | | | | | | |

SPTB models with gene expression only

| | | | | | | | | |
|---|---|---|---|---|---|---|---|---|
| A | — | 0.703 | 44.3 | 81.5 | 52.5 | 76.0 | 18.5 | 55.7 |
| B | — | 0.748 | 46.2 | 86.5 | 59.6 | 79.0 | 13.5 | 53.8 |
| C | — | 0.758 | 52.6 | 84.3 | 58.7 | 80.7 | 15.7 | 47.4 |

Area under receiver operator curve (ROC AUC)
*anaemia is defined as <120 g/L of haemoglobin

TABLE 4

| | Entrez Gene | | |
|---|---|---|---|
| | ID* (homo | Direction of | |
| HGNC Symbol | sapiens) | Change | Gene Annotation |
| ZNF605 | 100289635 | Down | Zinc finger protein 605 |
| LRRC41 | 10489 | Down | Leucine repeat containing 41 |
| PCDHGA12 | 26025 | Down | protocadherin gamma subfamily A, 12 |
| ABT1 | 29777 | Down | activator of basal transcription 1 |
| THBS3 | 7059 | Up | thrombospondin 3 |
| VNN1 | 8876 | Up | vanin 1 |
| LOC100128908 | 100128908 | Up | Leishmanolysin homolog |
| CST13P | 164380 | Up | Cystatin 13 pseudogene |
| EFF1D | 1936 | Down | Eukaryotic translation elongation factor 1 |
| | | | delta |
| RPH3A | 22895 | Up | Rabphilin 3A |
| TRBV6-6 | 28601 | Down | T cell receptor beta variable 6-6 |
| PLEC | 5339 | Up | plectin |
| MIR601 | 693186 | Up | microRNA 601 |
| ZNF16 | 7564 | Down | zinc finger protein 16 |
| MIR3691 | 100500900 | Up | microRNA 3691 |
| LOC101927441 | 101927441 | Down | uncharacterized LOC101927441 |
| ACAP2 | 23527 | Down | ArfGAP with coiled-coil, ankyrin repeat |
| | | | and PH domains 2 |
| ZNF324 | 25799 | Down | zinc finger protein 324 |
| SH3PXD2B | 285590 | Up | SH3 and PX domains 2B |
| TBX21 | 30009 | Up | T-box 21 |

*https://www.ncbi.nlm.nih.gov/gene

### BELOW ARE FULL CITATIONS FOR PUBLICATIONS MENTIONED HEREIN

Abbott, D. S., N. L. Hezelgrave, P. T. Seed, J. E. Norman, A. L. David, P. R. Bennett, J. C. Girling, M. Chandirimani, S. J. Stock, J. Carter, R. Cate, J. Kurtzman, R. M. Tribe, and A. H. Shennan. 2015. ‘Quantitative fetal fibronectin to predict preterm birth in asymptomatic women at high risk’, Obstet Gynecol, 125: 1168-76.
Alfirevic, Z., J. Owen, E. Carreras Moratonas, A. N. Sharp, J. M. Szychowski, and M. Goya. 2013. ‘Vaginal progesterone, cerclage or cervical pessary for preventing preterm birth in asymptomatic singleton pregnant women with a history of preterm birth and a sonographic short cervix’, Ultrasound Obstet Gynecol, 41: 146-51.
Baig, S. A., N. Khan, T. Baqai, A. Fatima, S. A. Karim, and S. Aziz. 2013. ‘Preterm birth and its associated risk factors. A study at tertiary care hospitals of Karachi, Pakistan’, J Pak Med Assoc, 63: 414-8.
Berghella, V., A. O. Odibo, M. S. To, O. A. Rust, and S. M. Althuisius. 2005. ‘Cerclage for short cervix on ultrasonography: meta-analysis of trials using individual patient-level data’, Obstet Gynecol, 106: 181-9.
Blencowe, H., S. Cousens, D. Chou, M. Oestergaard, L. Say, A. B. Moller, M. Kinney, and J. Lawn. 2013. ‘Born too soon: the global epidemiology of 15 million preterm births’, Reprod Health, 10 Suppl 1: S2.
Care, A. G., A. N. Sharp, S. Lane, D. Roberts, L. Watkins, and Z. Alfirevic. 2014. ‘Predicting preterm birth in women with previous preterm birth and cervical length >/=25 mm’, Ultrasound Obstet Gynecol, 43: 681-6.
da Fonseca, E. B., R. E. Bittar, M. H. Carvalho, and M. Zugaib. 2003. ‘Prophylactic administration of progesterone by vaginal suppository to reduce the incidence of spontaneous preterm birth in women at increased risk: a randomized placebo-controlled double-blind study’, Am J Obstet Gynecol, 188: 419-24.
Dai, M., P. Wang, A. D. Boyd, G. Kostov, B. Athey, E. G. Jones, W. E. Bunney, R. M. Myers, T. P. Speed, H. Akil, S. J. Watson, and F. Meng. 2005. ‘Evolving gene/transcript definitions significantly alter the interpretation of GeneChip data’, Nucleic Acids Res, 33: e175.
Dallas, P. B., N. G. Gottardo, M. J. Firth, A. H. Beesley, K. Hoffmann, P. A. Terry, J. R. Freitas, J. M. Boag, A. J. Cummings, and U. R. Kees. 2005. ‘Gene expression levels assessed by oligonucleotide microarray analysis and quantitative real-time RT-PCR —how well do they correlate?’, BMC Genomics, 6: 59.
DeFranco, E. A., J. M. O'Brien, C. D. Adair, D. F. Lewis, D. R. Hall, S. Fusey, P. Soma-Pillay, K. Porter, H. How, R. Schakis, D. Eller, Y. Trivedi, G. Vanburen, M. Khandelwal, K. Trofatter, D. Vidyadhari, J. Vijayaraghavan, J. Weeks, B. Dattel, E. Newton, C. Chazotte, G. Valenzuela, P. Calda, M. Bsharat, and G. W. Creasy. 2007. ‘Vaginal progesterone is associated with a decrease in risk for early preterm birth and improved neonatal outcome in women with a short cervix: a secondary analysis from a randomized, double-blind, placebo-controlled trial’, Ultrasound Obstet Gynecol, 30: 697-705.
Dekker, G. A., S. Y. Lee, R. A. North, L. M. McCowan, N. A. Simpson, and C. T. Roberts. 2012. ‘Risk factors for preterm birth in an international prospective cohort of nulliparous women’, PLoS One, 7: e39154.
Duhig, K. E., M. Chandiramani, P. T. Seed, A. L. Briley, A. P. Kenyon, and A. H. Shennan. 2009. ‘Fetal fibronectin as a predictor of spontaneous preterm labour in asymptomatic women with a cervical cerclage’, BJOG., 116: 799-803.
Enquobahrie, D. A., M. A. Williams, C. Qiu, S. Y. Muhie, K. Slentz-Kesler, Z. Ge, and T. Sorenson. 2009. ‘Early pregnancy peripheral blood gene expression and risk of preterm delivery: a nested case control study’, BMC Pregnancy Childbirth, 9: 56.
Fonseca, E. B., E. Celik, M. Parra, M. Singh, and K. H. Nicolaides. 2007. ‘Progesterone and the risk of preterm birth among women with a short cervix’, N Engl J Med, 357: 462-9.
Fortunato, S. J, and R Menon. 2003. ‘IL-1 beta is a better inducer of apoptosis in human fetal membranes than IL-6’, Placenta, 24: 922-28.
Friebe-Hoffmann, U, J. P Chiao, and P. N Rauk. 2001. ‘Effect of IL-lbeta and IL-6 on oxytocin secretion in human uterine smooth muscle cells’, Am J Reprod Immunol, 46: 226-31.
Gilbert, N. M., V. P. O'Brien, S. Hultgren, G. Macones, W. G. Lewis, and A. L. Lewis. 2013. ‘Urinary tract infection as a preventable cause of pregnancy complications: opportunities, challenges, and a global call to action’, Glob Adv Health Med, 2: 59-69.
Goya, M., L. Pratcorona, C. Merced, C. Rodo, L. Valle, A. Romero, M. Juan, A. Rodriguez, B. Munoz, B. Santacruz, J. C. Bello-Munoz, E. Llurba, T. Higueras, L. Cabero, and E. Carreras. 2012. ‘Cervical pessary in pregnant women with a short cervix (PECEP): an open-label randomised controlled trial’, Lancet, 379: 1800-6.
Gracie, S. K., A. W. Lyon, H. L. Kehler, C. E. Pennell, S. M. Dolan, D. A. McNeil, J. E. Siever, S. W. McDonald, A. D. Bocking, S. J. Lye, K. M. Hegadoren, D. M. Olson, and S. C. Tough. 2010. ‘All Our Babies Cohort Study: recruitment of a cohort to predict women at risk of preterm birth through the examination of gene expression profiles and the environment’, BMC Pregnancy Childbirth, 10: 87.
Heng, Y. J., C. E. Pennell, H. N. Chua, J. E. Perkins, and S. J. Lye. 2014. ‘Whole blood gene expression profile associated with spontaneous preterm birth in women with threatened preterm labor’, PLoS One, 9: e96901.
Heng, Y. J., L. Taylor, B. G. Larsen, H. N. Chua, S. M. Pung, M. W. Lee, M. Tucholska, S. Tate, P. Kupchak, C. E. Pennell, T. Pawson, and S. J. Lye. 2015. ‘Albumin decrease is associated with spontaneous preterm delivery within 48 h in women with threatened preterm labor’, J Proteome Res, 14: 457-66.
Honest, H, L. M Bachamann, J. K Gupta, J Kleijnen, and K. S. Khan. 2002. ‘Accuracy of cervicovaginal fetal fibronectin test in predicting risk of spontaneous preterm birth: systematic review’, BMJ, 325: 301.
Howson, C. P., M. V. Kinney, L. McDougall, and J. E. Lawn. 2013. ‘Born too soon: preterm birth matters’, Reprod Health, 10 Suppl 1: 51.
Hutzal, C. E., E. M. Boyle, S. L. Kenyon, J. V. Nash, S. Winsor, D. J. Taylor, and H. Kirpalani. 2008. ‘Use of antibiotics for the treatment of preterm parturition and prevention of neonatal morbidity: a metaanalysis’, Am J Obstet Gynecol, 199: 620 e1-8.
Institute of Medicine. 2007. Preterm birth. Causes, consequences, and prevention (National Academies Press: Washington DC).
Irizarry, R. A., B. Hobbs, F. Collin, Y. D. Beazer-Barclay, K. J. Antonellis, U. Scherf, and T. P. Speed. 2003. ‘Exploration, normalization, and summaries of high density oligonucleotide array probe level data’, Biostatistics, 4: 249-64.
Johansson, A. M., E. Domellof, and L. Ronnqvist. 2014. ‘Long-term influences of a preterm birth on movement organization and side specialization in children at 4-8 years of age’, Dev Psychobiol.
Kennard, E. A., P. D. Zimmerman, C. I. Friedman, and D. A. Kniss. 1995. ‘Interleukin-1 beta induces cyclooxygenase-2 in cultured human decidual cells’, Am J Reprod Immunol, 34: 65-71.
Kuhrt, K., E. Smout, N. Hezelgrave, P. T. Seed, J. Carter, and A. H. Shennan. 2016. ‘Development and validation of a tool incorporating cervical length and quantitative fetal fibronectin to predict spontaneous preterm birth in asymptomatic high-risk women’, Ultrasound Obstet Gynecol, 47: 104-9.
Leung, B. M., S. W. McDonald, B. J. Kaplan, G. F. Giesbrecht, and S. C. Tough. 2013. ‘Comparison of sample characteristics in two pregnancy cohorts: community-based versus population-based recruitment methods’, BMC Med Res Methodol, 13: 149.
Liggins, G. C. 1989. ‘Cervical ripening as an inflammatory reaction.’ in D. A Ellwood, A. B. M Anderson and M. P Embrey (eds.), The cervix in pregnancy and labour (Churchill-Livingstone: Edinburgh).
Makhlouf, M. A., R. G. Clifton, J. M. Roberts, L. Myatt, J. C. Hauth, K. J. Leveno, M. W. Varner, J. M. Thorp, Jr., B. M. Mercer, A. M. Peaceman, S. M. Ramin, J. D. Jams, A. Sciscione, J. E. Tolosa, and Y. Sorokin. 2014. ‘Adverse Pregnancy Outcomes among Women with Prior Spontaneous or Induced Abortions’, Am J Perinatol, 31: 765-72.
Martin, Joyce A., Brady E. Hamilton, Michelle J. K. Osterman, Sally C. Curtin, and T. J. Mathews. 2013. ‘Births: Final data for 2012’, National Vital Statistics Reports, 62.
McDonald, S. W., A. W. Lyon, K. M. Benzies, D. A. McNeil, S. J. Lye, S. M. Dolan, C. E. Pennell, A. D. Bocking, and S. C. Tough. 2013. ‘The All Our Babies pregnancy cohort: design, methods, and participant characteristics’, BMC Pregnancy Childbirth, 13 Suppl 1: S2.
Meis, P. J, M Klebanoff, E Thom, M. P Dombrowski, B Sibai, A. H Moawad, C. Y. Spong, J. C. Hauth, M. Miodovnik, M. W. Varner, K. J Leveno, S. N. Caritis, J. D Jams, and et al. 2003. ‘Prevention of recurrent preterm delivery by 17 alpha-hydroxyprogesterone caproate’, N Engl J Med, 348: 2379-85.
Menon, R., M. R. Torloni, C. Voltolini, M. Torricelli, M. Merialdi, A. P. Betran, M. Widmer, T. Allen, I. Davydova, Z. Khodjaeva, P. Thorsen, M. Kacerovsky, V. Tambor, T. Massinen, J. Nace, and C. Arora. 2011. ‘Biomarkers of spontaneous preterm birth: an overview of the literature in the last four decades’, Reprod Sci, 18: 1046-70.
Morey, J. S., J. C. Ryan, and F. M. Van Dolah. 2006. ‘Microarray validation: factors influencing correlation between oligonucleotide microarrays and real-time PCR’, Biol Proced Online, 8: 175-93.
Osman, I, A Young, F Jordan, L A Greer, and J. E Norman. 2006. ‘Leukocyte density and proinflammatory mediator expression in regional human fetal membranes and decidua before and during labor at term’, J Soc Gynecol Investig, 13: 97-103.
Osman, I, A Young, M Ledingham, A. J Thomson, F Jordan, L A Greer, and J. E Norman. 2003. ‘Leukocyte density and pro-inflammatory cytokine expression in human fetal membranes, decidua, cervix and myometrium before and during labour at term’, Mol Hum Reprod, 9: 41-45.
Pennell, C. E., B. Jacobsson, S. M. Williams, R. M. Buus, L. J. Muglia, S. M. Dolan, N. H. Morken, H. Ozcelik, S. J. Lye, and C. Relton. 2007. ‘Genetic epidemiologic studies of preterm birth: guidelines for research’, Am JObstet Gynecol, 196: 107-18.
Public Health Agency of Canada. 2012. “Perinatal Health Indicators for Canada 2011.” In. Ottawa, ON.
Räisänen, S, M. Gissler, J. Saari, M. Kramer, and S. Heinonen. 2013. ‘Contribution of risk factors to extremely, very and moderately preterm births—register-based analysis of 1,390,742 singleton births’, PLoS One, 8: e60660.
Rauk, P. N, and J. P Chiao. 2000. ‘Interleukin-1 stimulates human uterine prostaglandin production through induction of cyclooxygenase-2 expression’, Am J Reprod Immunol, 43: 152-59.
Romero, R., S. Durum, C. A. Dinarello, E. Oyarzun, J. C. Hobbins, and M. D. Mitchell. 1989. ‘Interleukin-1 stimulates prostaglandin biosynthesis by human amnion’, Prostaglandins, 37: 13-22.
Schmitz, T, M. J Leroy, E Dallot, M Breuiller-Fouche, F Ferre, and D Cabrol. 2003. ‘Interleukin-lbeta induces glycosaminoglycan synthesis via prostaglandin E2 pathway in cultured human cervical fibroblasts’, Mol Hum Reprod, 9: 1-8.
Simcox, R., P. T. Seed, P. Bennett, T. G. Teoh, L. Poston, and A. H. Shennan. 2009. ‘A randomized controlled trial of cervical scanning vs history to determine cerclage in women at high risk of preterm birth (CIRCLE trial)’, Am JObstet Gynecol, 200: 623 e1-6.
Sing, T., O. Sander, N. Beerenwinkel, and T. Lengauer. 2005. ‘ROCR: visualizing classifier performance in R’, Bioinformatics, 21: 3940-1.
Smyth, G. K. 2004. ‘Linear models and empirical bayes methods for assessing differential expression in microarray experiments’, Stat Appl Genet Mol Biol, 3: Article3.
Subramanian, A., P. Tamayo, V. K. Mootha, S. Mukherjee, B. L. Ebert, M. A. Gillette, A. Paulovich, S. L. Pomeroy, T. R. Golub, E. S. Lander, and J. P. Mesirov. 2005. ‘Gene set enrichment analysis: a knowledge-based approach for interpreting genome-wide expression profiles’, Proc Natl Acad Sci USA, 102: 15545-50.
Thomson, A. J, J. F Telfer, A Young, S Campbell, C. J Stewart, I. T Cameron, L A Greer, and J. E Norman. 1999. ‘Leukocytes infiltrate the myometrium during human parturition: further evidence that labour is an inflammatory process’, Hum Reprod, 14: 229-36.
Treyvaud, K., K. J. Lee, L. W. Doyle, and P. J. Anderson. 2014. ‘Very preterm birth influences parental mental health and family outcomes seven years after birth’, J Pediatr, 164: 515-21.
Ulmsten, U., L. Wingerup, P. Belfrage, G. Ekman, and N. Wiqvist. 1982. ‘Intracervical application of prostaglandin gel for induction of term labor’, Obstet Gynecol, 59: 336-9.
van den Broek, N. R., R. Jean-Baptiste, and J. P. Neilson. 2014. ‘Factors associated with preterm, early preterm and late preterm birth in Malawi’, PLoS One, 9: e90128.
Vandesompele, J., K. De Preter, F. Pattyn, B. Poppe, N. Van Roy, A. De Paepe, and F. Speleman. 2002. ‘Accurate normalization of real-time quantitative RT-PCR data by geometric averaging of multiple internal control genes’, Genome Biol, 3: research0034.
Watari, M, H Watari, M. E DiSanto, S Chacko, G. P Shi, and J. F Strauss 3rd. 1999. ‘Pro-inflammatory cytokines induce expression of matrix-metabolizing enzymes in human cervical smooth muscle cells’, Am J Pathol, 154: 1755-62.
Word, R. A., X. H. Li, M. Hnat, and K. Carrick. 2007. ‘Dynamics of cervical remodeling during pregnancy and parturition: mechanisms and current concepts’, Semin Reprod Med., 25: 69-79.
Yi, S. W., Y. J. Han, and H. Ohrr. 2013. ‘Anemia before pregnancy and risk of preterm birth, low birth weight and small-for-gestational-age birth in Korean women’, Eur J Clin Nutr, 67: 337-42.

What is claimed is:

1. A method for detecting spontaneous preterm birth in an asymptomatic subject comprising: (a) subjecting a sample from the subject to a procedure to detect polynucleotides or polypeptides encoded by the polynucleotides in the sample; (b) detecting spontaneous preterm birth by comparing the amount of polynucleotides or polypeptides encoded by the polynucleotides to the amount of such polynucleotides or polypeptides obtained from a control who does not suffer from preterm birth wherein the polynucleotides comprise at least one of, or are selected from ZNF605, LRRC41, PCDHGA12, ABT1, THBS3, VNN1, LOC100128908, CST13P, EEF1D, RPH3A, TRBV6-6, PLEC, MIR601, ZNF16, MIR3691, LOC101927441, ACAP2, ZNF324, SH3PXD2B, and TBX21.

2. A method of claim 1 wherein the polynucleotides comprise ZNF605, LRRC41, PCDHGA12, ABT1, THBS3 and VNN1.

3. A method of claim 1 wherein the polynucleotides comprise LOC100128908, CST13P, EEF1D, RPH3A, TRBV6-6, PLEC, MIR601, and ZNF16.

4. A method of claim 1 wherein the polynucleotides comprise LOC100128908, MIR3691, LOC101927441, CST13P, ACAP2, ZNF324, SH3PXD2B and TBX21.

5. A method of claim 1 wherein the procedure comprises detecting one or more polynucleotides in the sample by contacting the sample with oligonucleotides that hybridize to the polynucleotides; and detecting in the sample levels of nucleic acids that hybridize to the polynucleotides relative to a control, wherein a change or significant difference in the amount or status of the polynucleotides in the sample compared with the amount or status in the control is indicative of spontaneous preterm birth.

6. A method of claim 1 wherein the procedure comprises:
(a) contacting the sample with antibodies that specifically bind to the polypeptides under conditions effective to bind the antibodies and form complexes;
(b) measuring the amount or status of the polypeptides present in the sample by quantitating the amount of the complexes; and
(c) wherein a change or significant difference in the amount or status of polypeptides in the sample compared with the amount or status obtained from a control subject who does not suffer from preterm birth is indicative of spontaneous preterm birth.

7. A method of claim 1 wherein the sample is maternal peripheral blood.

8. A panel of biomarkers for diagnosing or monitoring preterm birth comprising (a) ZNF605, LRRC41, PCDHGA12, ABT1, THBS3, VNN1, LOC100128908, CST13P, EEF1D, RPH3A, TRBV6-6, PLEC, MIR601, ZNF16, MIR3691, LOC101927441, ACAP2, ZNF324, SH3PXD2B and TBX2; (b) ZNF605, LRRC41, PCDHGA12, ABT1, THBS3 and VNN1, (c). LOC100128908, CST13P, EEF1D, RPH3A, TRBV6-6, PLEC, MIR601 and ZNF16, or (d) LOC100128908, MIR3691, LOC101927441, CST13P, ACAP2, ZNF324, SH3PXD2B and TBX21, or polypeptides encoded by (a), (b), (c), or (d).

9. A method of diagnosing spontaneous preterm birth in an asymptomatic subject, the method comprising measuring the level of each biomarker of a panel of biomarkers of claim 8 in a sample from the subject, wherein each biomarker of the panel of biomarkers is measured using a respective reagent that specifically measures the biomarker.

10. A method of claim 9 wherein the sample is maternal peripheral blood.

11. A method for monitoring the progression of pregnancy that results in spontaneous preterm birth in a subject, the method comprising: (a) detecting in a sample from the subject at a first time point, each biomarker of a panel of biomarkers of claim 8; (b) repeating step (a) at a subsequent point in time; and (c) comparing levels detected in steps (a) and (b), and thereby monitoring the progression of pregnancy that results in preterm birth.

12. A method of claim 11 wherein the sample is maternal peripheral blood.

13. A kit for carrying out a method of claim 1.

14. A kit comprising reagents capable of hybridizing to, and/or measuring the expression of, at least one of the biomarkers ZNF605, LRRC41, PCDHGA12, ABT1, THBS3, VNN1, LOC100128908, CST13P, EEF1D, RPH3A, TRBV6-6, PLEC, MIR601, ZNF16, MIR3691, LOC101927441, ACAP2, ZNF324, SH3PXD2B, and TBX21.

15. A kit of claim 14 wherein the biomarkers comprise ZNF605, LRRC41, PCDHGA12, ABT1, THBS3 and VNN1.

16. A kit of claim 14 wherein the biomarkers comprise LOC100128908, CST13P, EEF1D, RPH3A, TRBV6-6, PLEC, MIR601, and ZNF16.

17. A kit of claim 14 wherein the biomarkers comprise LOC100128908, MIR3691, LOC101927441, CST13P, ACAP2, ZNF324, SH3PXD2B and TBX21.

18. A kit of claim 14, wherein the reagents are polynucleotide probes which are labelled with a detectable substance.

19. A kit of claim 14, wherein the reagents are polypeptides which are labelled with a detectable substance.


SEQUENCE LISTING (38 sequences)

SEQ ID NO: 1 - DNA, 20 bp, Artificial Sequence
  primer
gccctgctgtggaagaattg

SEQ ID NO: 2 - DNA, 22 bp, Artificial Sequence
  primer
aacaggatacaaggccagaaga

SEQ ID NO: 3 - DNA, 21 bp, Artificial Sequence
  primer
ctggtggggaagtggaagatt

SEQ ID NO: 4 - DNA, 21 bp, Artificial Sequence
  primer
ccaaacgttggaacaaagcct

SEQ ID NO: 5 - DNA, 20 bp, Artificial Sequence
  primer
ctggaagtggagaaacccga

SEQ ID NO: 6 - DNA, 20 bp, Artificial Sequence
  primer
gctacagaagccaagcgact

SEQ ID NO: 7 - DNA, 20 bp, Artificial Sequence
  primer
acaggtgccatggatgtagc

SEQ ID NO: 8 - DNA, 20 bp, Artificial Sequence
  primer
ctgtttcaatgctggcctcg

SEQ ID NO: 9 - DNA, 20 bp, Artificial Sequence
  primer
cctgccatggaatcccctac

SEQ ID NO: 10 - DNA, 21 bp, Artificial Sequence
  primer
ttctgagggactgctaacacg

SEQ ID NO: 11 - DNA, 21 bp, Artificial Sequence
  primer
ccgctacatctatggcccaat

SEQ ID NO: 12 - DNA, 20 bp, Artificial Sequence
  primer
cccaggtcataagcccagtc

SEQ ID NO: 13 - DNA, 22 bp, Artificial Sequence
  primer
tgagaaaggactcctcgtccat

SEQ ID NO: 14 - DNA, 20 bp, Artificial Sequence
  primer
accgagtccctcatgatgct

SEQ ID NO: 15 - DNA, 20 bp, Artificial Sequence
  primer
agcacaggctttgaccgata

SEQ ID NO: 16 - DNA, 20 bp, Artificial Sequence
  primer
gctcgcaattacggggtttt

SEQ ID NO: 17 - DNA, 22 bp, Artificial Sequence
  primer
cagctggaggtggagataagaa

SEQ ID NO: 18 - DNA, 20 bp, Artificial Sequence
  primer
ccacgatttctcggcgaatg

SEQ ID NO: 19 - DNA, 20 bp, Artificial Sequence
  primer
cttgctgccattctcctggt

SEQ ID NO: 20 - DNA, 22 bp, Artificial Sequence
  primer
catgtttttccttgagcctgga

SEQ ID NO: 21 - DNA, 21 bp, Artificial Sequence
  primer
tgctcttcaggtttcaggctc

SEQ ID NO: 22 - DNA, 20 bp, Artificial Sequence
  primer
gcgtgcagcagtatgtgaaa

SEQ ID NO: 23 - DNA, 24 bp, Artificial Sequence
  primer
tcgtgtcaaccccaaatttttacg

SEQ ID NO: 24 - DNA, 25 bp, Artificial Sequence
  primer
acaaagagttgtgttaaagatgctg

SEQ ID NO: 25 - DNA, 20 bp, Artificial Sequence
  primer
cctgccatggaatcccctac

SEQ ID NO: 26 - DNA, 21 bp, Artificial Sequence
  primer
ttctgagggactgctaacacg

SEQ ID NO: 27 - DNA, 21 bp, Artificial Sequence
  primer
ccgctacatctatggcccaat

SEQ ID NO: 28 - DNA, 20 bp, Artificial Sequence
  primer
cccaggtcataagcccagtc

SEQ ID NO: 29 - DNA, 20 bp, Artificial Sequence
  primer
gtcaaagttgtggtcgctgt

SEQ ID NO: 30 - DNA, 20 bp, Artificial Sequence
  primer
ttagctccgcccttcaaagt

SEQ ID NO: 31 - DNA, 22 bp, Artificial Sequence
  primer
tgagaaaggactcctcgtccat

SEQ ID NO: 32 - DNA, 20 bp, Artificial Sequence
  primer
accgagtccctcatgatgct

SEQ ID NO: 33 - DNA, 20 bp, Artificial Sequence
  primer
tgggaacaagagggcatctg

SEQ ID NO: 34 - DNA, 22 bp, Artificial Sequence
  primer
ccaccactgcatcaaattcatg

SEQ ID NO: 35 - DNA, 21 bp, Artificial Sequence
  primer
tgcacaggagccaagagtgaa

SEQ ID NO: 36 - DNA, 20 bp, Artificial Sequence
  primer
cacatcacagctccccacca

SEQ ID NO: 37 - DNA, 24 bp, Artificial Sequence
  primer
acttttggtacattgtggcttcaa

SEQ ID NO: 38 - DNA, 20 bp, Artificial Sequence
  primer
ccgccaggacaaaccagtat